(19) United States
(12) Patent Application Publication
(10) Pub. No.: US 2021/0353639 A1
(43) Pub. Date: Nov. 18, 2021

(54) MAST1 AND USES FOR DIAGNOSING AND TREATING CANCER

(71) Applicant: EMORY UNIVERSITY, Atlanta, GA (US)

(72) Inventors: Sumin KANG, Atlanta, GA (US); Jing CHEN, Atlanta, GA (US); Lingtao JIN, Atlanta, GA (US)

(21) Appl. No.: 16/963,454

(22) PCT Filed: Jan. 18, 2019
(86) PCT No.: PCT/US19/14305

§ 371 Date: Jul. 20, 2020

Related U.S. Application Data
(60) Provisional application No. 62/619,004, filed on Jan. 18, 2018.

(51) Int. Cl. A61K 31/553; A61P 35/00; A61K 45/06; A61K 31/337; A61K 33/243

(52) U.S. Cl. CPC A61K 31/553; A61P 35/00; A61K 33/243; A61K 31/337; A61K 45/06

(57) ABSTRACT

Described are methods of treating cancer comprising administering an effective amount of a platinum-based chemotherapy agent in combination with a microtubule associated serine/threonine-protein kinase (MAST) inhibitor, e.g., MAST1, and/or other kinase inhibitor to a subject in need thereof. Also described are methods of detecting amounts of MAST1 in a sample thereby determining whether the subject is sensitive or resistant to a platinum-based chemotherapy agent or combination of chemotherapy agents comprising the same. Kits comprising a platinum-based chemotherapy agent and a microtubule associated serine/threonine-protein kinase (MAST) inhibitor are also described.

### CROSS REFERENCE TO RELATED APPLICATIONS

This application claims the benefit of priority to U.S. Provisional Application No. 62/619,004, filed Jan. 18, 2018, which is incorporated by reference herein in its entirety.

### ACKNOWLEDGEMENT OF GOVERNMENT FUNDING

This invention was made with government support under grant no. CA207768 awarded by the National Institutes of Health. The government has certain rights in this invention.

### BACKGROUND

Platinum-based chemotherapy is employed for the treatment of a wide array of solid malignancies including head and neck, lung, and ovarian cancers. Cisplatin and other similar platinum-based drugs lead to an initial therapeutic success, but many patients have tumors that are intrinsically resistant or develop resistance to cisplatin treatment. Cisplatin exerts anti-cancer effects mainly by interacting with DNA to form mostly intrastrand crosslink adducts, which activate pro-apoptotic signal transduction pathways. Cisplatin resistance likely occurs due to complex reasons, including increased drug efflux, drug breakdown, increased repair of damaged DNA, and increased activation of pro-survival pathways or inhibition of pathways that promote cell death. Although a group of signaling effectors have been identified as predictive markers of cisplatin resistance including MRP2, ERCC1, ATPase7A/7B/11B, ERBB2, Bcl-2, and survivin, most of these studies lacked either an assessment of clinical correlation or an explanation for how these protein factors regulate pro-survival signals in the presence of cisplatin. Therefore, the detailed molecular mechanisms of platinum-based drug resistance still remain elusive.

Protein kinases are often involved in pro-survival signaling pathways. The serine/threonine kinase microtubule-associated serine/threonine-protein kinase 1 (MAST1, also known as SAST170) belongs to a family containing four members, MAST1-MAST4. MAST family members share approximately 49%-64% sequence homology and contain four distinct domains including DUF1908, serine/threonine kinase domain, AGC-kinase C-terminal domain, and PDZ domain. MAST1 is reported to function as a scaffold protein to link the dystrophin/utrophin network with microfilaments via syntrophin. Recurrent rearrangement of the MAST1 gene has been observed in breast cancer cell lines and tissues. Nonetheless, little is known about the biological role of MAST1 as a kinase and its role in human cancers.

Higher MEK1 expression in cancers is associated with platinum-based drug resistance and correlates with shortened progression-free survival of patients. Activation of the MAPK family of proteins has been implicated in response to platinum-based chemotherapy. For instance, inhibition of MEK/ERK signaling augmented cisplatin sensitivity in human squamous cell carcinoma. Although the importance of MEK in cancer and its contribution to chemotherapy response is well studied, the detailed molecular mechanisms by which MEK is activated in response to platinum-based drug treatment, and how it consequently contributes to cisplatin response, is unclear.

What are thus needed are new compositions and methods for addressing cisplatin and other platinum-based drug resistance in cancers. Further, what are needed are new methods of chemosensitizing therapeutic targets for the treatment of patients with platinum-based chemotherapy resistant cancer. The disclosed compositions and methods address these and other needs.

### SUMMARY

This disclosure relates, in certain aspects, to methods of treating cancer comprising administering an effective amount of a platinum-based chemotherapy agent in combination with a microtubule associated serine/threonine-protein kinase 1 (MAST1) inhibitor and/or other kinase inhibitor to a subject in need thereof. In certain embodiments, this disclosure relates to methods of detecting amounts of MAST1 in a sample to determine or monitor whether the subject is sensitive or resistant to a platinum-based chemotherapy agent or combination of chemotherapy agents comprising the same.

In certain embodiments, the MAST1 inhibitor is a MAST1 specific binding agent such as an antibody, small molecule compound, peptide, or siRNA. In certain embodiments, the MAST1 inhibitor is lestaurtinib, a derivative, a prodrug, or a salt thereof.

In certain embodiments, this disclosure relates to methods of detecting amounts of MAST1 in a sample to determine whether the subject is sensitive or resistant, or is going to develop resistance to a platinum-based chemotherapy agent or combination of chemotherapy agents comprising the same. In certain embodiments, the sample is derived from tumor cells or cell-free DNA contained in a blood sample of a subject diagnosed with cancer.

In certain embodiments, this disclosure relates to methods for diagnosing and treating cancer in a subject comprising: a) obtaining a sample from a human subject with cancer, wherein the subject has been administered a platinum-based chemotherapy agent; b) detecting elevated MAST1 protein or encoding nucleic acid in the sample from the subject with cancer compared to a control, wherein the control amount is determined from a cancerous sample considered sensitive to a platinum-based chemotherapy agent; c) diagnosing the subject as a subject with cancer that is resistant to the platinum-based chemotherapy agent; and d) administering an effective amount of the microtubule associated serine/threonine-protein kinase 1 (MAST1) inhibitor or other kinase inhibitor or in combination with the platinum-based chemotherapy agent to the subject.

In certain embodiments, diagnosis is not needed. For example, in one embodiment, a method is provided, said method comprising: administering an effective amount of the microtubule associated serine/threonine-protein kinase 1 (MAST1) inhibitor, or other kinase inhibitor, in combination with a platinum-based chemotherapy agent to a subject. Optionally, elevated MAST1 protein or encoding nucleic acid is detected in the sample from a subject with cancer compared to a control.

In certain embodiments, the methods disclosed herein further comprise administering an additional anti-cancer agent.

In certain embodiments, this disclosure relates to medicaments comprising a platinum-based chemotherapy agent in combination with a microtubule associated serine/threonine-protein kinase 1 (MAST1) inhibitor for use in treating cancer as provided herein. In certain embodiments, this disclosure relates to medicaments comprising a platinum-based chemotherapy agent and a medicament with a microtubule associated serine/threonine-protein kinase 1 (MAST1) inhibitor for use in treating cancer as provided herein.

In certain embodiments, this disclosure relates to a use of an effective amount of a platinum-based chemotherapy agent in combination with a microtubule associated serine/threonine-protein kinase (MAST1) inhibitor in the preparation of a medicament for a cancer that is resistant to the platinum-based chemotherapy agent without the MAST1 inhibitor.

In certain embodiments, this disclosure provides a kit comprising an effective amount of a combination of a platinum-based chemotherapy agent and a microtubule associated serine/threonine-protein kinase (MAST) inhibitor. The effective amount is sufficient to provide a synergistic response in a subject who has a cancer that is resistant to the platinum-based chemotherapy agent without the MAST1 inhibitor.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIG. 1A illustrates RNAi screens use to identify MAST1 as a synthetic lethal target for cisplatin treatment in human cancers. Primary screen testing 781 genes was carried out using sublethal dose (5 μg/ml) of cisplatin.

FIG. 1B illustrates a secondary screen used the top 50 candidates from the primary screen in 4 cisplatin-resistant cancer cell lines (KB-3-1^{cisR}, A549^{cisR}, A2780^{cisR} and PCI-15A^{cisR}). There were thirty (30) leads showing >10% cell death upon shRNA and cisplatin treatment.

FIG. 1C shows data where KB-3-1^{cisR} and A549^{cisR} cells were transduced with 3 different MAST1 shRNA clones followed by sublethal dose of cisplatin (5 μg/ml for KB-3-1^{cisR} and 2 μg/ml for A549^{cisR}) or vehicle treatment.

FIG. 1D shows cell viability and cisplatin sensitivity of platinum-refractory (cis^{R}) SCLC patient-derived tumor TKO-002 cells with MAST1 knockdown.

FIG. 1E shows data where colony formation assays were performed using cancer cells with MAST1 knockdown and cisplatin treatment.

FIG. 1F shows data on the effect of cisplatin treatment and MAST1 knockdown using two shRNA clones on tumor growth of KB-3-1^{cisR} xenograft mice. Mice were treated with vehicle-control or cisplatin and tumor size was monitored.

FIG. 1G shows data where tumor weight was examined at the experimental endpoint.

FIG. 1H shows data on MAST1 expression in tumor lysates. Representative images of Ki-67 IHC staining in harvested tumors from each group are shown. The data in FIGS. 1C-1H indicate targeting MAST1 sensitizes cisplatin treatment in vitro and in vivo.

FIG. 2A shows data for a MAST1 in vitro kinase assay using MAST1 wild type (WT) or kinase-dead mutant (DA; D497A). GST-MAST1 variants were enriched from 293T and kinase activity was assessed by ADP-Glo Kinase assay using myelin basic protein (MBP) as a substrate.

FIG. 2B shows cell viability of parental cancer cells with MAST1 WT or DA overexpression in the presence of cisplatin.

FIG. 2C shows cell viability of cisplatin-resistant (cis^{R}) cells with rescue expression of shRNA-resistant MAST1 WT or DA and endogenous MAST1 knockdown. Relative viability was obtained by normalizing values to cisplatin untreated samples.

FIG. 2D shows phosphorylation sites showing phosphorylation levels most decreased by MAST1 knockdown and cisplatin treatment. Phospho-antibody array results were obtained using 1,318 antibodies in KB-3-1^{cisR} lysates.

FIG. 2E shows data when parental and cis^{R} pairs of KB-3-1 and A549 cells with MAST1 knockdown and cisplatin treatment (5 μg/ml) were assayed for MEK1 phosphorylation by immunoblotting.

FIG. 2F shows data for an MAST1 in vitro kinase assay using recombinant inactive MEK1 (rMEK1 K79A) as a substrate.

FIG. 2G shows data for kinase activities of MEK1, cRaf and MAST1 in cells with MAST1 knockdown and cisplatin treatment. MEK1, cRaf and MAST1 were immunoprecipitated from KB-3-1 cells and kinase activities were determined by ADP-Glo kinase assay using recombinant inactive ERK1 or MEK1 as substrates. MEK inhibitor U0126 (10 μM) and Raf inhibitor L779450 (5 μM) were used as controls.

FIG. 2H shows western blot analysis of apoptosis-related factors. Cells with or without MAST1 shRNA were treated with cisplatin (5 μg/ml) for 16 hours.

FIG. 2I shows data for an apoptosis assay using parental and cis^{R} cells with or without MAST1 knockdown. Cells were treated with sublethal dose of cisplatin (2 μg/ml: parental, 5 μg/ml: cis^{R}) for 48 hours and apoptotic cells were assayed by annexin V staining. This data indicates MAST1 directly phosphorylates MEK1 and activates anti-apoptotic signaling upon cisplatin treatment.

FIG. 3A shows data indicating dissociation of cRaf and MEK1 upon cisplatin treatment in diverse cancer types. Cells were treated with 5 μg/ml cisplatin for 24 hours prior to MEK1 immunoprecipitation.

FIG. 3B shows data indicating in vitro MEK1-cRaf dissociation. Purified MEK1-cRaf complex from KB-3-1 cells was resuspended in TBS buffer, incubated with increasing concentrations of cisplatin (0-5 μg/ml) for 2 hours, and applied to Western blotting.

FIG. 3C indicates MAST1 interacts with cRaf and MEK1 in cancer cells. ov: overexpressed.

FIG. 3D shows data on the effect of cRaf or MAST1 downregulation on MEK1 activation in the presence and absence of cisplatin. cRaf and MAST1 knockdown cells were treated with cisplatin for 48 hours and MEK1 activity was determined by p-MEK S217/S221 Western blotting.

FIG. 3E shows data on the effect of MAST1 rescue expression on MEK1 activation in MAST1 knockdown cells with cisplatin treatment.

FIG. 3F shows data on the effect of MAST1 WT or kinase-dead DA rescue expression on MEK1 activation in cRaf knockdown cells in the presence or absence of cisplatin.

FIG. 3G shows data indicating cisplatin-mediated in vitro MEK1-cRaf dissociation. Purified MEK1-cRaf complex from KB-3-1 cells was resuspended in TBS buffer and incubated with 5 μg/ml cisplatin. Samples were collected at different time points and subjected to immunoblotting. Density analysis of relative amount of cRaf bound to MEK1 from three biological replicates are shown.

FIG. 3H shows data where an accumulation of cisplatin-induced DNA damage and repair in KB-3-1 cells with or without MAST1 shRNA was determined by flow cytometry analysis of phospho-γH2AX and phospho-53BP1. For DNA repair, cells were treated with cisplatin (5 μg/ml) for 2 hours before cisplatin washed off and cells were incubated in fresh medium.

FIG. 3I show data where Cisplatin-DNA adduct accumulation and removal was analyzed by flow cytometry or Inductively Coupled Plasma Mass Spectrometry (ICP-MS) in KB-3-1 cells with or without MAST1 shRNA.

FIG. 3J shows data indicating cRaf-MEK1 dissociation induced by DNA damaging agents and cytotoxic drugs. KB-3-1 cells were treated with different compounds as indicated, followed by MEK1 immunoprecipitation and immunoblotting.

FIG. 3K shows data where cells with or without MAST1 knockdown were treated with different concentrations of DNA damaging agents and cytotoxic drugs and IC₅₀ values were calculated using GraphPad Prism 6. This data indicates cisplatin, but not the other DNA damaging agents, dissociates cRaf from MEK1 and reactivates MEK1 through MAST1.

FIG. 4A shows data where MAST1, flag-tagged MEK1 S221A and S221D expression were detected by immunoblotting in KB-3-1^{cisR}, A549^{cisR} and KB-3-1 cells.

FIG. 4B shows cell viability in MAST1 knockdown cells with MEK1 variants in the presence and absence of sublethal dose of cisplatin (2 μg/ml: parental, 5 μg/ml: cis^{R}).

FIG. 4C shows data on apoptosis.

FIG. 4D shows data on tumor volume, tumor weight, and Ki-67 expression of KB-3-1^{cisR} cell xenograft mice. Representative dissected tumors are shown on right top panel. KB-3-1^{cisR} cells with MAST1 knockdown and MEK1 S221A or S221D expression were injected and cisplatin was administered by intraperitoneal injection. Tumor volume and tumor weight were normalized to the corresponding cisplatin untreated group. This data indicates MAST1 induces cisplatin-resistant cancer cell proliferation and tumor growth through MEK1 phosphorylation.

FIG. 5A shows data on MAST1 expression in HNSCC, lung, and ovarian cancer cell lines.

FIG. 5B shows data indicating a correlation between MAST1 expression and cisplatin IC₅₀ in cancer cell lines shown in 5A.

FIG. 5C shows data on MAST1 expression in patient-derived tumors.

FIG. 5D shows data indicating a correlation between MAST1 expression and cisplatin IC₅₀ in patient-derived tumors shown in 5C. r=Pearson's correlation coefficient.

FIG. 5E shows data on the effect of MAST1 knockdown on cisplatin response in MAST1 expressing 212LN and UDSCC2 cells.

FIG. 5F shows data on the effect of MAST1 WT or kinase-dead mutant DA overexpression on cisplatin resistance in low MAST1 expressing MDA686TU and Tu-212 cells. Cisplatin response was determined using cisplatin IC₅₀. This data indicates MAST1 expression correlates with cisplatin resistance in cancer cell lines and patient-derived tumors.

FIG. 6A shows a schematic summary of HNSCC patients (n=97) and samples (n=116) analyzed by IHC. Sensitive: patients with no evidence of disease for two years after platinum therapy; Resistant: patients with tumor recurrence within two years of platinum therapy. Open circle: pre-therapy tumors; closed circle: post-therapy tumors.

FIG. 6B shows date on MAST1 expression and MEK phosphorylation levels and drug response in pre-platinum treatment specimens.

FIG. 6C shows data for post-platinum treatment specimens.

FIG. 6D shows data on the correlation between MAST1 and phospho-MEK in pre20 treatment and post-treatment samples.

FIG. 6E shows data comparisons of MAST1 status between pre- and post-therapy in paired samples.

FIG. 6F shows data on non-paired samples.

FIG. 6G shows a Kaplan-Meier survival analysis of a platinum-treated patient groups.

FIG. 6H shows data for the non-platinum-treated patient group. Patients were dichotomized by MAST1 expression level at median. This data indicates MAST1 and MEK activation is associated with cisplatin resistance and poor clinical outcome in human HNSCC.

FIG. 7A shows from an in vitro MAST1 kinase assay using ten drugs potentially bind to MAST1. 10 μM drugs were incubated with purified GST-MAST1 and applied to the in vitro kinase assay. In addition to lestaurtinib, five additional compounds dovitinib, staurosporine, sunitinib, SU14813, and bosutinib inhibited MAST1 activity in vitro. Among them, lestuartinib sensitized cisplatin treatment the most in cancer cells.

FIG. 7B show data on the effect of top six MAST1 inhibitors from (7A) on cisplatin sensitivity in KB-3-1 cells. A dose of inhibitor that did not affect viability when given alone (100 nM of lestaurtinib, dovitinib, staurosporine; 1 μM of sunitinib, SU14813, bosutinib) was used with increasing concentrations of cisplatin for 48 hours.

FIG. 7C shows data indicating lestaurtinib inhibits MAST1 activity in vitro (left) and in vivo in KB-3-1 cells (right).

FIG. 7D shows a cellular thermal shift assay using KB-3-1 cells harboring MAST1 WT or L504D treated with lestaurtinib.

FIG. 7E shows data on kinase activity of MAST1 WT or L504D treated with lestaurtinib. Purified GST-MAST1 variants were treated with increasing concentrations of lestaurtinib.

FIG. 7F shows cell viability and cisplatin IC₅₀ upon lestaurtinib treatment in KB-3-1 cells with endogenous MAST1 knockdown expressing MAST1 WT or L504D.

FIG. 7G show data on the effect of lestaurtinib on MAST1 activity at a range of ATP concentrations.

FIG. 7H shows Cell viability and cisplatin IC₅₀ upon lestaurtinib treatment in KB-3-1 (top) and 212LN (bottom) cells with or without MAST1 knockdown.

FIG. 7I shows data on the effect of MAST1 knockdown on tumor growth of lestaurtinib and cisplatin treated mice carrying KB-3-1^{cisR} cell xenografts. Error bars represent SEM. Representative dissected tumors for each group are shown. Scale bar represents 5 mm.

FIG. 7J shows tumor weight at the experimental endpoint. Error bars represent SD.

FIG. 7K shows data on Ki-67 expression determined by IHC staining. MAST1 expression in tumor lysates is shown. Scale bar represents 50 μm.

FIG. 8A shows cell viability, colony formation assay, apoptosis induction, and MEK1 phosphorylation in KB-3-1 and 212LN with vehicle control, lestaurtinib, cisplatin and the combination. Combination Index (CI) value for cell viability was obtained by CompuSyn.

FIG. 8B shows data on how lestaurtinib effects cell viability and cisplatin sensitivity of SCLC patient-derived tumor TKO-002. CI value for synergistic effect is shown.

FIG. 8C shows data on the effect of lestaurtinib, cisplatin and the combination effect on tumor growth of cis^{R} HNSCC (left), lung cancer (middle), and ovarian cancer (right) PDX mice. Pt-8 tumor and Pt-11 tumor in FIG. 5C were used for lung cancer PDX and ovarian cancer PDX, respectively. Error bars represent SEM. Scale bars for the dissected tumors represent 5 mm.

FIG. 8D shows data for Ki-67 expression determined by IHC staining Scale bars represent 50 μm.

FIG. 8E shows data where MAST1 activity was assessed by MAST1 in vitro kinase assay using MBP as a substrate.

FIG. 8F shows data on MEK1 inhibition by lestaurtinib and cisplatin combination in PDX tumor lysates. MEK activity was assessed by phospho-S217/S221 MEK immunoblotting. This data indicates lestaurtinib sensitizes cancer cells to cisplatin treatment in vitro and in vivo.

FIG. 8G illustrates a proposed model for some embodiments involving the role of MAST1 in cisplatin resistance in human cancer. This model is not intended to be limiting. Cancer cells rely on cRaf-dependent MEK1 activation to promote proliferation and tumor growth in the absence of cisplatin. Cisplatin treatment dissociates cRaf from MEK1, while MAST1 phosphorylates MEK1 to activate the MAPK pathway in cRaf-independent manner, inhibiting BIM and providing a proliferative advantage to cancer cells. Targeting MAST1 by lestaurtinib restores cisplatin sensitivity to cells.

FIG. 9 shows data on tumor growth size indicating lestaurtinib or trametinib sensitizes cancer cells to cisplatin in patient-derived xenograft (PDX) mice. Pt-9 tumor in FIG. 5C was used for PDX. Lung PDX mice were treated with vehicle control, lestaurtinib (20 mg/kg intraperitoneal injection, 5 times a week), trametinib (1 mg/kg oral gavage, daily), cisplatin (5 mg/kg intraperitoneal injection, twice a week), or the combinations.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. To the extent that any term is defined in any of the references that have been incorporated by reference, it is the use and definition of the term as provided herein that controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

### General Definitions

In this specification and in the claims that follow, reference will be made to a number of terms, which shall be defined to have the following meanings:

As used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. patent law in that they are inclusive or open-ended and do not exclude additional, unrecited elements or method steps. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps composition components or method steps, etc., that do not materially affect the basic and novel characteristic(s) of the compositions or methods, compared to those of the corresponding compositions or methods disclosed herein.

Reference to “MAST1” protein refers to the microtubule-associated serine/threonine protein kinase 1 [Homo sapiens] with Genbank NCBI Reference Sequence: NP_055790.1 (SEQ ID NO.:1). A MAST1 inhibitor refers to a specific binding agent for MAST1 that has the effect of decreasing kinase activity in vivo or in vitro. Alternative names for MAST1 include Syntrophin-associated serine/threonine-protein kinase (SAST).

A “specific binding agent” may be a protein, peptide, nucleic acid, carbohydrate, lipid, or small molecular weight compound that specifically binds to the MAST1 protein. In certain embodiments, the specific binding agent according to the present disclosure is an antibody or binding fragment thereof (e.g., Fab, F(ab′)₂), peptide or a peptibody, or MAST1 binding fragments thereof. WO00/24782 and WO03/057134 (incorporated herein by reference) describe and teach making binding agents that contain a randomly generated peptide which binds a desired target. A specific binding agent can be a proteinaceous polymeric molecule (a “large molecule”) such as an antibody or Fc-peptide fusion, or a non-proteinaceous non-polymeric molecule typically having a molecular weight of less than about 1200 Daltons (a “small molecule”).

The term “specifically binds” refers to the ability of a specific binding agent of the present invention, under specific binding conditions, to bind a target molecule such that its affinity is at least times as great, but optionally 50 times as great, 100, 250 or 500 times as great, or even at least 1000 times as great as the average affinity of the same specific binding agent to a large collection of random peptides or polypeptides. A specific binding agent need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a molecule having a substantially similar epitope as the target molecule (e.g., a paralog) is within the scope of the term “specific binding” which is determined relative to a statistically valid sampling of unique non-targets (e.g., random polypeptides).

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

As used herein, the term “cancer” refers to a malignant neoplasm (Stedman's Medical Dictionary, 25th ed.; Hensly ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor Tlymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

As used herein a “sample” refers to a composition taken from or originating from a subject. Examples of samples include cell samples, tumor cells, blood samples, tissue samples, hair samples, semen, and urine or excrement samples. Additional examples include tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals. In certain embodiments, the subject is a human.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression. In some embodiments, “treat” denotes the therapeutic treatment of a disease, for example, in a subject with current symptoms. In some embodiments, “treat” denotes the prophylactic treatment for the prevention of a disease.

An “effective amount” refers to an amount sufficient to elicit the desired biological response, i.e., treating the condition. As will be appreciated by those of ordinary skill in this art, the effective amount may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount may reduce the tumor burden or stop the growth or spread of a tumor. In some embodiments, “effective amount” denotes the amount of two or more components, that, when combined is sufficient to elicit the desired biological response. This subset of effective amount can be denoted as a “combined effective amount.” In some embodiments, the “effective amount” can be an amount sufficient for each ingredient, when considered separately, to provide a desired biological response.

As used herein, the term “combination with” when used to describe administration with an additional treatment means that the agent may be administered prior to, together with, or after the additional treatment, or a combination thereof.

A “chemotherapy agent,” “chemotherapeutic,” “anti-cancer agent” or the like, refer to molecules that are recognized to aid in the treatment of a cancer. Contemplated examples include the following molecules or derivatives such as alemtuzumab, trastuzumab, ibritumomab tiuxetan, brentuximab vedotin, temozolomide, ado-trastuzumab emtansine, denileukin diftitox, blinatumomab, interferon alpha, aldesleukin, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin, rituximab, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

For example, if a disclosed compound or a pharmaceutically acceptable form of the compound contains a carboxylic acid functional group, a prodrug can comprise a pharmaceutically acceptable ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as betadimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

If a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆)alkanoyloxy) ethyl, 1-methyl-1((C₁-C₆)alkanoyloxy)ethyl (C₁-C₆)alkoxycarbonyloxymethyl, —N—(C₁-C₆)alkoxycarbonylaminomethyl, succinoyl, (C₁-C₆)alkanoyl, alpha-amino(C₁-C₄)alkanoyl, arylacyl and alpha-aminoacyl, or alpha-aminoacyl-alpha-aminoacyl, where each alpha-aminoacyl group is independently selected from naturally occurring L-amino acids P(O)(OH)₂, —P(O)(O(C₁-C₆)alkyl)₂, and glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, a natural alpha-aminoacyl, —C(OH)C(O)OY₁ wherein Y₁ is H, (C₁-C₆)alkyl or benzyl, —C(OY₂)Y₃ wherein Y₂ is (C₁-C₄) alkyl and Y₃ is (C₁-C₆)alkyl, carboxy(C₁-C₆)alkyl, amino(C₁-C₄)alkyl or mono-Nor di-N,N—(C₁-C₆)alkylaminoalkyl, —C(Y₄)Y₅ wherein Y₄ is H or methyl and Y₅ is mono-N- or di-N,N—(C₁-C₆)alkylamino, morpholino, piperidin-1-yl or pyrrolidin-1-yl.

As used herein, “pharmaceutically acceptable esters” include, but are not limited to, alkyl, alkenyl, alkynyl, aryl, arylalkyl, and cycloalkyl esters of acidic groups, including, but not limited to, carboxylic acids, phosphoric acids, phosphinic acids, sulfonic acids, sulfinic acids, and boronic acids.

As used herein, “pharmaceutically acceptable enol ethers” include, but are not limited to, derivatives of formula —C═C(OR) where R can be selected from alkyl, alkenyl, alkynyl, aryl, aralkyl, and cycloalkyl. Pharmaceutically acceptable enol esters include, but are not limited to, derivatives of formula —C═C(OC(O)R) where R can be selected from hydrogen, alkyl, alkenyl, alkynyl, aryl, aralkyl, and cycloalkyl.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom or replacing an amino group with a hydroxyl group. The derivative may be a prodrug. Derivatives may be prepared by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NR^{a}R^{b}, —NR^{a}C(═O)R^{b}, —NR^{a}C(═O)NR^{a}NR^{b}, —NR^{a}C(═O)OR^{b}, —NR^{a}SO₂R^{b}, —C(═O)R^{a}, —C(═O)OR^{a}, —C(═O)NR^{a}R^{b}, —OC(═O)NR^{a}R^{b}, —OR^{a}, —SR^{a}, —SOR^{a}, —S(═O)₂R^{a}, —OS(═O)₂R^{a} and —S(═O)₂OR^{a}. R^{a} and R^{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

Control levels can be used to establish a threshold value, e.g., such that a value greater than the threshold value indicates the subject has increased MAST1 proteins or encoding nucleic acids. This threshold value can be determined empirically by comparing positive controls (samples from subjects with cancer or a particular type of cancer, e.g., cisplatin resistant) and negative controls (samples of subjects without cancer or who have been successfully treated for cancer, e.g., cisplatin sensitive). Such controls are optionally age matched or matched according to cancer type or stage. In order to distinguish elevated MAST1 values, the threshold value can be set at least 1, 1.5, 2, 2.5, 3, 3.5, 4, 4.5, or 5 standard deviations above the mean negative control value. Other statistical methods can be used to set a threshold value that is within the desired predictive power needed for the assay. For example, the threshold value can be set such that there is no statistically significant difference between the threshold value and the positive control values using routine statistical analysis.

The terms “cutoff” and “cutoff value” mean a value measured in an assay that defines the dividing line between two subsets of a population (e.g., responders and non-responders). Thus, a value that is equal to or higher than the cutoff value defines one subset of the population; and a value that is lower than the cutoff value defines the other subset of the population. The terms “threshold” and “threshold level” mean a level that defines the dividing line between two subsets of a population (e.g., responders and non-responders). A threshold level may be a prevalence cutoff or a cutoff value.

MAST1 Drives Cisplatin Resistance in Human Cancers by Rewiring cRaf Independent MEK Activation

Experiments reported herein provide insights into cancer cisplatin resistance by characterizing the role of MAST1 in driving cisplatin resistance and its mode of action in human cancers involving rewiring of the MEK signaling pathway. While not intending to be limited by the model, these findings support a clinical strategy to enhance the susceptibility of cancers to cisplatin. The kinase inhibitor lestaurtinib is a potent MAST1 inhibitor and cisplatin sensitizing agent. Studies reported herein indicate a mechanism by which MAST1 contributes to the development of cisplatin resistance via the MAPK pathway. Using MAST1 inhibitors is provided as a method to battle cisplatin and other platinum-based chemotherapeutic resistant cancers.

To identify protein kinases that are uniquely required for chemotherapy resistance, synthetic lethal screening experiments were performed using an shRNA library targeting 781 human kinases and kinase-related genes. Through a series of screening cycles using head and neck, lung, and ovarian cancer cells treated with two of the most commonly used chemotherapy agents, cisplatin and taxol, a serine/threonine kinase microtubule associated serine/threonine-protein kinase 1 (MAST1, a.k.a. SAST170), was identified as specifically contributing to cisplatin resistance in diverse types of cancers by signaling through MEK. MAST1 belongs to a family containing four members, MAST1-MAST4. MAST family members share approximately 49-64% sequence homology and contain four distinct domains including DUF1908, serine/threonine kinase domain, AGC-kinase C-terminal domain, and PDZ domain MAST1 is reported to function as a scaffold protein to link the dystrophin/utrophin network with microfilaments via syntrophin. Recurrent gene rearrangement of MAST1 has been observed in breast cancer cell lines and tissues. Very little was known about the biological role of MAST1 as a kinase and its role in human cancers.

Activation of the MAPK family of proteins has been implicated in response to platinum-based chemotherapy. MEK1 expression positively correlates with shortened progression free survival in human cancer and is associated with platinum-based drug resistance. For instance, inhibition of MEK/ERK signaling augmented cisplatin sensitivity in human squamous cell carcinoma. Detailed molecular mechanisms by which MEK is activated in response to platinum-based drug treatment, and how it consequently contributes to cisplatin response, was not previously determined. Experiments reported herein indicate that cisplatin inhibits the cRaf-MEK pathway and that reactivation of MAST1-dependent MAPKs provides cisplatin resistance in human cancers. Thus, based on the disclosure provided herein, MAST1 can be targeted for cancer therapy in combination with cisplatin or other platinum-based chemotherapy agent. It has been discovered that the kinase inhibitor lestaurtinib is a potent MAST1 inhibitor and a chemosensitizing agent for clinical treatment of patients with cisplatin-resistant cancer.

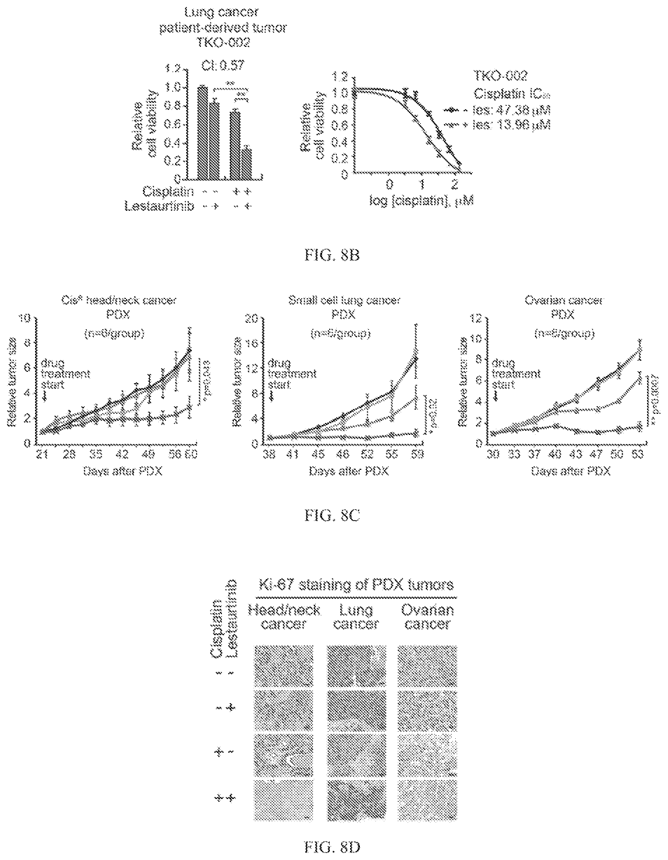

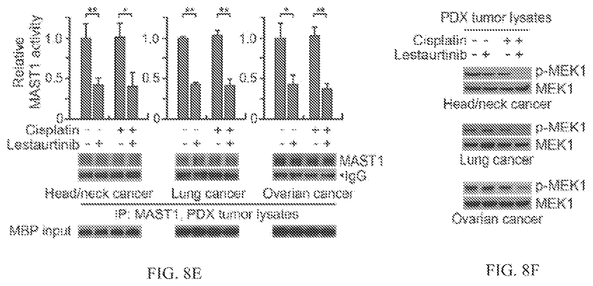

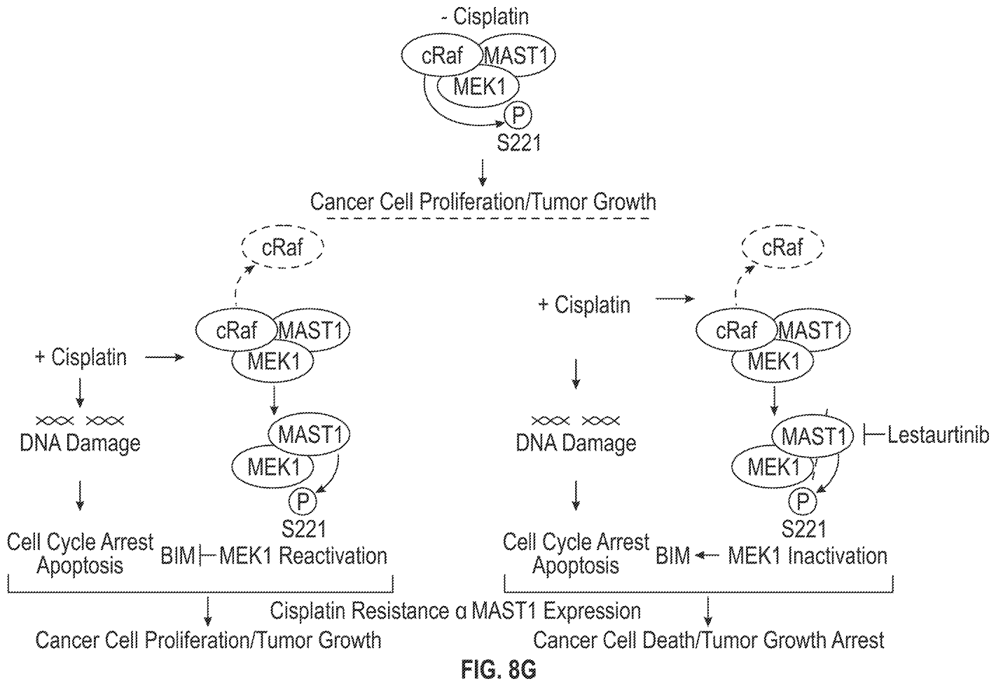

Although it is not intended that embodiments of this disclosure be limited by any particular mechanism, it is believed that MAST1 as a lethal partner of cisplatin that functions as a factor to program cisplatin-resistant pro-survival signaling in human cancers. Experiments reported herein indicate a mechanistic basis by which MAST1 controls cancer cells to evade cisplatin-induced cell death. Cells predominantly depend on Raf for MEK1 activation during active cell proliferation. Consistently, although MAST1 is present in the cRaf-MEK1 complex, cancer cells depend on cRaf-dependent MEK1 activation to promote proliferation and tumor growth (FIG. 8G). However, cisplatin treatment dissociates cRaf from MEK1, whereas MAST1 remains in complex with MEK1. MAST1 phosphorylates MEK1, leading to cRaf-independent activation of MEK1 and the downstream MAPK pathway including loss of the pro-apoptotic BIM. This MAST1-mediated MEK1 activation provides anti-apoptotic and proliferative protection to cancer cells treated with cisplatin, which, if sufficient to reverse the pro-apoptotic signaling induced by cisplatin, promotes cancer cell proliferation and tumor growth (FIG. 8G). Studies using clinical samples supports that upregulation of MAST1 positively correlates with nonresponse to platinum therapy. There may exist two complementary paths to MAST1-mediated platinum resistance, which are initially high levels of MAST1 and the ability to upregulate MAST1 in response to platinum.

From a clinical perspective, experiments reported herein support that MAST1 serves as a predictive marker and as a therapeutic target to treat cancer patients in combination with platinum-based chemotherapy. Thus, a mature small molecule kinase inhibitor lestaurtinib was used as a potent MAST1 inhibitor with cisplatin sensitization activity. Lestaurtinib was originally reported as a tyrosine kinase inhibitor that inhibits FLT3 as well as JAK2 and Trk. It was determined that lestaurtinib functions as a multi-kinase inhibitor and effectively inhibits the serine/threonine kinase MAST1. Other FLT3, JAK2, and Trk inhibitors did not confer cisplatin sensitization and lestaurtinib had no effect on cisplatin response in cells deficient in MAST1 suggest that MAST1 represents the primary target through which lestaurtinib sensitizes cancer cells to cisplatin. Lestaurtinib was under clinical trial evaluation. Lestaurtinib was effective in abolishing cisplatin resistance in patient-derived xenografts of diverse cancers, which indicates that this approach can be commonly applied to treat various types of cancers. In some embodiments, MAST1 targeted therapy is even more beneficial to patients with advanced cancers or patients who received platinum-based therapy but recurred, in part, due to the induction of MAST1 during the treatment.

### Methods of Treatment and Diagnosis

This disclosure relates to methods of treating cancer comprising administering an effective amount of a platinum-based chemotherapy agent in combination with a microtubule associated serine/threonine-protein kinase 1 (MAST1) inhibitor and/or other kinase inhibitor(s) to a subject in need thereof. The administration of the MAST1 inhibitor can be prior to, concurrent with, or after administration of the platinum-based chemotherapy agent.

In certain embodiments, the MAST1 inhibitor is a MAST1 specific binding agent such as an antibody, small molecule compound, peptide, or siRNA.

In certain embodiments, the MAST1 inhibitor is lestaurtinib, derivative, prodrug, or salts thereof. In certain embodiments, the platinum-based chemotherapy agent is selected from cisplatin, carboplatin, oxaliplatin, phenanthriplatin, nedaplatin, triplatin tetranitrate, picoplatin, pyriplatin, lipoplatin, and satraplatin.

In certain embodiments, the cancer is selected from testicular cancer, ovarian cancer, cervical cancer, breast cancer, bladder cancer, head and neck cancer, esophageal cancer, lung cancer, mesothelioma, brain tumors and neuroblastoma. In certain embodiments, the subject is at risk of, exhibiting symptoms or diagnosed with the cancer. In certain embodiments, the cancer is skin cancer, bladder cancer, breast cancer, colon cancer, rectal cancer, endometrial cancer, kidney cancer, leukemia, liver cancer, lung cancer, melanoma, Hodgkin and non-Hodgkin lymphoma, pancreatic cancer, prostate cancer, thyroid cancer, glioblastoma, or brain cancer. In certain embodiments, the lung cancer is small cell lung cancer (SCLC) or non-small cell lung cancer (NSCLC). In certain embodiments, the lung cancer is metastatic non-small cell lung cancer (NSCLC) with BRAF V600E mutation.

In certain embodiments, this disclosure relates to methods of diagnosing cancer sensitive or resistant to a platinum-based chemotherapy agent in an individual comprising: providing a biological sample from the individual, and detecting the amount of MAST1 protein or nucleic within said sample, wherein increased amounts of MAST1 protein or nucleic acid in said biological sample compared to control, reference or a sample obtained from a cancerous or non-cancerous sample considered sensitive to a platinum-based chemotherapy agent, indicates that said biological sample is resistant to a platinum-based chemotherapy agent. In certain embodiments, the biological sample or reference sample is determined to be abnormal, malignant, pre-malignant or combinations thereof. In some embodiments, the control is a negative control, and the control is from a sample that is sensitive to a platinum-based chemotherapy agent. In some embodiments, the control is a positive control, and can be compared to a level in a sample that is known to be resistant to a platinum-based chemotherapy agent.

In certain embodiments, this disclosure relates to methods for diagnosing a subject during a chemotherapy treatment comprising detecting an amount MAST1 protein or nucleic acid encoding MAST1 in a sample from the subject and correlating the amount to whether the subject is sensitive to or resistant to a platinum-based chemotherapy agent, wherein the subject is considered resistant to the platinum-based chemotherapy agent if the amount is higher than control, reference, or normal sample, and wherein the subject is considered sensitive to the to the platinum-based chemotherapy agent if the amount is similar to the control, reference, or normal sample. In certain embodiments, the methods further comprise administering a MAST1 inhibitor and/or another chemotherapy agent or kinase inhibitor if the subject is considered resistant to the platinum-based chemotherapy agent.

In certain embodiments, this disclosure relates to methods of diagnosing a platinum-based chemotherapy resistant tumor in an individual comprising: providing a biological sample from the individual, and detecting the amount of MAST1 protein or nucleic within said sample, wherein increased amounts of MAST1 protein or nucleic acid in said biological sample compared to a sample obtained from a platinum-based chemotherapy-sensitive tumor indicates that said biological sample is more platinum-based chemotherapy resistant than the platinum-based chemotherapy-sensitive tumor.

In certain embodiments, this disclosure relates to methods of diagnosing and treating a subject suffering from platinum-based chemotherapy resistant tumors, the method comprising: a) diagnosing whether the subject has a platinum-based chemotherapy resistant form of the disease based on a level of MAST1 protein or mRNA previously determined to be present in a sample of tumor cells from the subject and b) administering to the subject an amount effective of a MAST1 inhibitor or other kinase inhibitor, wherein the level of MAST1 protein or mRNA is equal to or above a pre-determined threshold.

In certain embodiments, this disclosure relates to methods for diagnosing and treating cancer in a subject comprising: a) obtaining a sample from a human subject with cancer, wherein the subject has been administered a platinum-based chemotherapy agent; b) detecting a MAST1 protein or nucleic acid encoding MAST1 the sample from the subject with cancer providing an amount of MAST1 protein or nucleic acid; c) diagnosing the subject as a subject with cancer that is responsive to a microtubule associated serine/threonine-protein kinase 1 (MAST1) inhibitor or other kinase inhibitor when the amount of MAST1 protein or encoding nucleic acid is in excess of a reference amount; d) administering an effective amount of the microtubule associated serine/threonine-protein kinase 1 (MAST1) inhibitor or other kinase inhibitor or in combination with the platinum-based chemotherapy agent to the subject diagnosed as responsive to the microtubule associated serine/threonine-protein kinase 1 (MAST1) inhibitor or other kinase inhibitor. In certain embodiments, the reference amount is determined from a cancerous sample considered sensitive to a platinum-based chemotherapy agent.

In certain embodiments, this disclosure relates to methods for diagnosing and treating cancer in a subject comprising: a) obtaining a sample from a subject with cancer, wherein the subject has been administered a platinum-based chemotherapy agent; b) detecting a MAST1 protein or nucleic acid encoding MAST1 the sample from the subject with cancer providing an amount of MAST1 protein or nucleic acid; c) diagnosing the subject as a subject with cancer that is resistant to the platinum-based chemotherapy agent when the amount of MAST1 protein or encoding nucleic acid is in excess of a normal, reference, or control amount; and d) administering an effective amount of an alternative chemotherapy. In certain embodiments, the alternative chemotherapy is a platinum-based chemotherapy agent in combination with a microtubule associated serine/threonine-protein kinase 1 (MAST1) inhibitor or other kinase inhibitor. In certain embodiments, the alternative chemotherapy does not contain a platinum-based chemotherapy agent.

In certain embodiments, this disclosure relates to methods for diagnosing a subject during a chemotherapy treatment comprising detecting an amount of MAST1 protein or nucleic acid encoding MAST1 in a sample from the subject and correlating the amount to whether the subject is sensitive to or resistant to a platinum-based chemotherapy agent, wherein the subject is considered resistant to the platinum-based chemotherapy agent if the amount is higher than a, control, reference, or normal sample, or the subject is considered sensitive to the to the platinum based chemotherapy agent if the amount is equivalent or does not exceed a control, reference or normal sample. In certain embodiments, the methods further comprise administering a MAST1 inhibitor and/or another or alternative chemotherapy agent(s) or kinase inhibitor to the subject if the subject is considered resistant to the platinum-based chemotherapy agent. In certain embodiments, the reference level is from a tumor sample determined to be sensitive to platinum-based chemotherapy agent.

In certain embodiments, the sample is from a tumor in in subject. In certain embodiments, the control amount is a measurement from a tumor that is determined to be sensitive to the platinum-based chemotherapy agent. In certain embodiments, the normal amount is an amount detected in a sample that is not diseased or cancerous. In certain embodiments, the alternative chemotherapy is a platinum-based chemotherapy agent in combination with a MAST1 inhibitor.

In certain embodiments, the kinase inhibitor is a MEK1 inhibitor such as trametinib (N-[3-[3-cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide), derivative prodrug, or salts thereof.

In certain embodiments, this disclosure relates to methods for diagnosing and treating cancer in a subject comprising: a) obtaining a sample from a subject with cancer, wherein the subject has been administered a platinum-based chemotherapy agent; b) detecting a MAST1 protein or nucleic acid encoding MAST1 the sample from the subject with cancer providing an amount of MAST1 protein or nucleic acid; c) diagnosing the subject as a subject with cancer that may be responsive to a microtubule associated serine/threonine-protein kinase 1 (MAST1) inhibitor or other kinase inhibitor when the amount of MAST1 protein or encoding nucleic acid is in excess of a normal, reference, or control amount; and d) administering an effective amount of the microtubule associated serine/threonine-protein kinase 1 (MAST1) inhibitor or other kinase inhibitor or in combination with the platinum-based chemotherapy agent to the subject diagnosed as likely responsive to the microtubule associated serine/threonine-protein kinase 1 (MAST1) inhibitor or other kinase inhibitor.

In some embodiments, the disclosure provides a method comprising: administering an effective amount of the microtubule associated serine/threonine-protein kinase 1 (MAST1) inhibitor, or other kinase inhibitor, in combination with a platinum-based chemotherapy agent to a subject. Optionally, elevated MAST1 protein or encoding nucleic acid is detected in the sample from a subject with cancer compared to a control.

In some embodiments, the disclosure provides a method comprising: receiving an effective amount of a microtubule associated serine/threonine-protein kinase 1 (MAST1) inhibitor, or other kinase inhibitor, in combination with a platinum-based chemotherapy agent. The subject receiving the combination is one who has a cancer or a tumor that is non-responsive to a platinum-based chemotherapy agent on its own.

In certain embodiments, this disclosure relates to methods of determining, prior to administering chemotherapy, whether a subject is going to be responsive or non-responsive to a platinum-based chemotherapy agent comprising detecting an amount of MAST1 protein or encoding nucleic acid in a sample of the subject and comparing the amount to a normal, reference, or control amount, wherein if the subject has elevated levels then administering a platinum-based chemotherapy agent in combination with a microtubule associated serine/threonine-protein kinase 1 (MAST1) inhibitor or administering alternative chemotherapy does not contain a platinum-based chemotherapy agent.

In certain embodiments, the alternative chemotherapy is selected from administering a combination therapy comprising cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); or rituximab, cyclophosphamide, doxorubicin, vincristine, prednisolone (RCHOP). In certain embodiments, the alternative chemotherapy is anti-CTLA4 (e.g., ipilimumab, tremelimumab) antibodies and/or the anti-PD1/PD-L1 (e.g., nivolumab, pidilizumab, pembrolizumab, atezolizumab, avelumab, durvalumab) antibodies.

In certain embodiments, the sample is from a tumor in in subject. In certain embodiments, the amount detected in a sample is abnormal, pre-malignant, or malignant and the sample is considered sensitive to a platinum-based chemotherapy agent or a reference level similar thereto.

In certain embodiments, this disclosure relates to methods for diagnosing, monitoring, and treating cancer in a subject comprising: a) obtaining a sample from a subject with cancer, wherein the subject has been administered a platinum-based chemotherapy agent; b) failing to detect MAST1 protein or nucleic acid in the sample or detecting a MAST1 protein or nucleic acid encoding MAST1 the sample from the subject with cancer providing an amount of MAST1 protein or nucleic acid; and c) diagnosing the subject as a subject with cancer that is not going to be responsive to a microtubule associated serine/threonine-protein kinase 1 (MAST1) inhibitor or other kinase inhibitor when the amount of MAST1 protein or encoding nucleic acid is of a normal, reference, or control amount.

In certain embodiments, this disclosure relates to methods for diagnosing, monitoring, and treating cancer in a subject comprising: a) obtaining a first sample from a subject with cancer, wherein the subject has been administered a platinum-based chemotherapy agent; b) failing to detect MAST1 protein or nucleic acid in the sample or detecting a MAST1 protein or nucleic acid encoding MAST1 the sample from the subject with cancer providing an amount of MAST1 protein or nucleic acid; c) diagnosing the subject as a subject with cancer that is not going to be responsive to a microtubule associated serine/threonine-protein kinase 1 (MAST1) inhibitor or other kinase inhibitor when the amount of MAST1 protein or encoding nucleic acid is of a normal, reference, or control amount; d) obtaining a second sample from the subject with cancer at a later time point than the first sample was obtained, wherein the subject has been administered a platinum-based chemotherapy agent; e) detecting a MAST1 protein or nucleic acid encoding MAST1 the sample from the subject with cancer providing an amount of MAST1 protein or nucleic acid; f) diagnosing the subject as a subject with cancer that is responsive to a microtubule associated serine/threonineprotein kinase 1 (MAST1) inhibitor or other kinase inhibitor when the amount of MAST1 protein or encoding nucleic acid is in excess of a normal, reference, or control amount; g) administering an effective amount of the microtubule associated serine/threonine-protein kinase 1 (MAST1) inhibitor or other kinase inhibitor or in combination with the platinum-based chemotherapy agent to the subject diagnosed as responsive to the microtubule associated serine/threonine-protein kinase 1 (MAST1) inhibitor or other kinase inhibitor. In certain embodiments, the MAST1 inhibitor is lestaurtinib, derivative, prodrug, or salts thereof.

In certain embodiments, the kinase inhibitor is a MEK1 inhibitor such as trametinib (N-[3-[3-cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide), derivative prodrug, or salts thereof.

In certain embodiments, this disclosure relates to method for diagnosis, detection or monitoring of a platinum-based chemotherapy in a tumor resistant or sensitive subject. In certain embodiments, a biological sample and/or a control/reference sample is from a tissue or organ corresponding to the tissue or organ which is to be diagnosed, detected or monitored with respect to affection by a tumor; e.g. the tumor which is to be diagnosed, detected or monitored is lung cancer and the biological sample and/or control/reference sample is lung tissue. Such tissues and organs are described herein, for example, in connection with different tumor diseases and cancers.

Preferably, the detection and/or determination of the quantity in the methods of the disclosure comprises (i) contacting a biological sample with an agent which binds specifically to the MAST1 protein or nucleic acid which is to be detected and/or the amount of which is to be determined, and (ii) detecting the formation of and/or determining the quantity of a complex between the agent and the MAST1 protein or nucleic acid which is to be detected or the amount of which is to be determined.

Typically, the level of the MAST1 in a biological sample is compared to a reference level, wherein a deviation from said reference level is indicative of the resistant and/or sensitivity to a platinum-based chemotherapy in a subject. A “deviation” from said reference level designates any significant change, such as an increase or decrease by at least 10%, 20%, or 30%, preferably by at least 40% or 50%, or even more.

Typically, the detection and/or determination of the quantity in the methods of the disclosure involves the use of labeled ligands which specifically bind to MAST1, e.g. a labeled nucleic acid probe that hybridizes to a MAST1 nucleic acid and/or a labeled antibody or fragment/derivative thereof that specifically binds to MAST1.

According to the disclosure, detection of a nucleic acid or determining the quantity of a nucleic acid may be carried out using known nucleic acid detection methods such as methods involving hybridization or nucleic acid amplification techniques. In one embodiment, mRNA transcripts are detected or the quantity thereof is determined using RT-PCR or Northern blot analysis.

### Combination Therapies

In certain embodiments, the methods disclosed herein further comprise administering an additional anti-cancer agent or alternative chemotherapy such as administering alemtuzumab, trastuzumab, ibritumomab tiuxetan, brentuximab vedotin, temozolomide, ado-trastuzumab emtansine, denileukin diftitox, blinatumomab, interferon alpha, aldesleukin, carmustine, bevacizumab, procarbazine, lomustine, vincristine, gefitinib, erlotinib, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin, rituximab, and/or lenalidomide or combinations thereof.

In certain embodiments, the methods disclosed herein further comprise administering an additional anti-cancer agent such as anti-CTLA4 (e.g., ipilimumab, tremelimumab) antibodies and/or the anti-PD1/PD-L1 (e.g., nivolumab, pidilizumab, pembrolizumab, atezolizumab, avelumab, durvalumab) antibodies.

In certain embodiments, the methods disclosed herein contemplate the subject is administered a combination of microtubule associated serine/threonine-protein kinase 1 (MAST1) inhibitor, a taxane, and a platinum-based chemotherapy agent. In certain embodiments, the taxane is paclitaxel, taxol, docetaxel, or combinations thereof. In certain embodiments, the subject is administered a combination of lestaurtinib, paclitaxel, and cisplatin.

In certain embodiments, the methods disclosed herein contemplate the subject is administered a combination of microtubule associated serine/threonine-protein kinase 1 (MAST1) inhibitor, bleomycin, etoposide, and a platinum-based chemotherapy agent.

In certain embodiments, the subject is administered a combination of lestaurtinib, bleomycin, etoposide, and cisplatin.

In certain embodiments, the methods disclosed herein contemplate the subject is administered a combination of microtubule associated serine/threonine-protein kinase 1 (MAST1) inhibitor, epirubicin, 5-fluorouracil and a platinum-based chemotherapy agent.

In certain embodiments, the subject is administered a combination of lestaurtinib, epirubicin, 5-fluorouracil, and cisplatin. In certain embodiments, the subject is administered a combination of lestaurtinib and cisplatin.

In certain embodiments, the methods disclosed herein contemplate the subject is administered a combination of microtubule associated serine/threonine-protein kinase 1 (MAST1) inhibitor, epirubicin, capecitabine, and a platinum-based chemotherapy agent. In certain embodiments, the subject is administered a combination of lestaurtinib, epirubicin, capecitabine, and cisplatin.

In certain embodiments, the methods disclosed herein contemplate the subject is administered a combination of microtubule associated serine/threonine-protein kinase 1 (MAST1) inhibitor, methotrexate, vincristine, doxorubicin, and a platinum-based chemotherapy agent. In certain embodiments, the subject is administered a combination of lestaurtinib, methotrexate, vincristine, doxorubicin, and cisplatin.

### MAST1 Inhibitors

In certain embodiments, a MAST1 inhibitor is a small molecule compound selected from lestaurtinib, dovitinib, midostaurin, bosutinib, sunitinib, neratinib, staurosporine, ruxolitinib, SU14813, or combinations thereof.

Davis et al. report analysis of kinase inhibitor selectivity. (Nat Biotechnol. 2011, 29(11):1046-51). In certain embodiments, the small molecule compound is lestaurtinib (7-hydroxy-7-(hydroxymethyl)-8-methyl-5,6,7,8,13,14-hexahydro-15H-16-oxa-4b,8a,14-triaza-5,8-methanodibenzo[b,h]cycloocta[jkl]cyclopenta[e]-as-indacen-15-one) (CAS Registry Number 111358-88-4), prodrugs, derivatives, or salts thereof.

In certain embodiments, the small molecule compound is dovitinib [4-amino-5-fluoro-3-(5-(4-methylpiperazin-1-yl)-1H-benzimidazol-2-yl)quinolin-2(1H)-one]), prodrugs, derivatives, or salts thereof.

In certain embodiments, the small molecule compound is midostaurin [N-(10-methoxy-9-methyl-1-oxo-2,3,10,11,12,13-hexahydro-9,13-epoxy-1H,9H-diindolo(1,2,3-GH:3′,2′,1′-1m)pyrrolo(3,4-j)(1,7)benzodiazonin-11-yl)-n-methylbenzamide]), prodrugs, derivatives, or salts thereof.

In certain embodiments, the small molecule compound is bosutinib (4-[(2,4-Dichloro-5-methoxyphenyl)amino]-6-methoxy-7-[3-(4-methyl-1-piperazinyl)propoxy]-3-quinolinecarbonitrile), prodrugs, derivatives, or salts thereof.

In certain embodiments, the small molecule compound is sunitinib [5-(5-fluoro-2-oxo-1,2-dihydroindolylidenemethyl)-2,4-dimethyl-1H-pyrrole-3-carboxylic acid (2-diethylaminoethyl)amide], prodrugs, derivatives, or salts thereof.

In certain embodiments, the small molecule is neratinib [N-(4-(3-chloro-4-(2-pyridinylmethoxy)anilino)-3-cyano-7-ethoxy-6-quinolyl)-4-(dimethylamino)-2-butenamide], prodrugs, derivatives, or salts thereof.

In certain embodiments, the small molecule compound is staurosporine (6-methoxy-5-methyl-7-methylamino-6,7,8,9,15,16-hexahydro-5H,14H-5,9-epoxy-4b,9a,15-triazadibenzo[b,h]cyclonona[1,2,3,4-jkl]cyclopenta[e]-as-indacen-14-one), prodrugs, derivatives, or salts thereof.

In certain embodiments, the small molecule compound is ruxolitinib [3-(4-(7h-pyrrolo[2,3-d]pyrimidin-4-yl)-1h-pyrazol-1-yl)-3-cyclopentylpropanenitrile], prodrugs, derivatives, or salts thereof.

In certain embodiments, the small molecule compound is SU14813 [5-((5-fluoro-2-oxo-1,2-dihydro-3H-indol-3-ylidene)methyl)-N-(2-hydroxy-3-morpholin-4-ylpropyl)-2,4-dimethyl-1H-pyrrole-3-carboxamide], prodrugs, derivatives, or salts thereof.

### Antibodies

In certain embodiments, this disclosure relates to uses of a MAST1 inhibitor that is a specific binding agent to MAST1 such as an antibody that binds MAST1.

The term “antibody” herein is used in the broadest sense and specifically covers full length monoclonal antibodies, immunoglobulins, polyclonal antibodies, multispecific antibodies (e.g. bispecific antibodies) formed from at least two full length antibodies, e.g., each to a different antigen or epitope, and individual antigen binding fragments, including dAbs, scFv, Fab, F(ab)′2, Fab′, including human, humanized and antibodies from non-human species and recombinant antigen binding forms such as monobodies and diabodies.

The term “human antibody” includes an antibody that possesses a sequence that is derived from a human germ-line immunoglobulin sequence, such as an antibody derived from transgenic mice having human immunoglobulin genes (e.g., XENOMOUSE genetically engineered mice (Abgenix, Fremont, Calif.), HUMAB-MOUSE™, KIRIN TC MOUSE™ transchromosome mice, KMMOUSE™ (MEDAREX, Princeton, N.J.)), human phage display libraries, human myeloma cells, or human B cells.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al. J. Mol. Biol., 222:581-597 (1991), for example.

The term “diabodies” refers to small antibody fragments with two antigen binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen binding sites. Diabodies are described more fully in, for example, EP 404,097; WO 93/11161; and Hollinger et al., Proc. Natl. Acad. Sci. USA, 90:6444-6448 (1993).

A “full length antibody” is one which comprises an antigen binding variable region as well as a light chain constant domain (CO and heavy chain constant domains, CH1, CH2 and CH3). The constant domains may be native sequence constant domains (e.g. human native sequence constant domains) or amino acid sequence variants thereof. In one aspect, the full length antibody has one or more effector functions.

A “glycosylation variant” antibody herein is an antibody with one or more carbohydrate moeities attached thereto which differ from one or more carbohydrate moieties attached to a main species antibody. Examples of glycosylation variants herein include antibody with a G1 or G2 oligosaccharide structure, instead of a G0 oligosaccharide structure, attached to an Fc region thereof, antibody with one or two carbohydrate moieties attached to one or two light chains thereof, antibody with no carbohydrate attached to one or two heavy chains of the antibody, etc, and combinations of glycosylation alterations.

Antibody “effector functions” refer to those biological activities attributable to the Fc region (a native sequence Fc region or amino acid sequence variant Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g. B cell receptor; BCR), and the like.

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The “light chains” of antibodies from any vertebrate species can be assigned to one of two clearly distinct types, called kappa and lambda, based on the amino acid sequences of their constant domains.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcgammaRIII only, whereas monocytes express FcgammaRI, FcgammaRII and FcgammaRIII. To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The terms “Fc receptor” or “FcR” are used to describe a receptor that binds to the Fc region of an antibody. In one aspect, the FcR is a native sequence human FcR. In another aspect, the FcR is one which binds an IgG antibody (a gamma receptor) and includes receptors of the FcgammaRI, FcgammaRII, and FcgammaRIII subclasses, including allelic variants and alternatively spliced forms of these receptors. FcgammaRII receptors include FcgammaRIIA (an “activating receptor”) and FcgammaRIIB (an “inhibiting receptor”), which have similar amino acid sequences that differ primarily in the cytoplasmic domains thereof. Activating receptor FcgammaRIIA contains an immunoreceptor tyrosine-based activation motif (ITAM) in its cytoplasmic domain. Inhibiting receptor FcgammaRIIB contains an immunoreceptor tyrosine-based inhibition motif (ITIM) in its cytoplasmic domain. (See review in M. Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:33-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “hypervariable region” when used herein refers to the amino acid residues of an antibody which are responsible for antigen binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk I Mol. Biol. 196:901-917 (1987)). “Framework Region” or “FR” residues are those variable domain residues other than the hypervariable region residues as herein defined. The hypervariable region or the CDRs thereof can be transferred from one antibody chain to another or to another protein to confer antigen binding specificity to the resulting (composite) antibody or binding protein.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

### RNA Interference

In certain embodiments, the MAST1 inhibitor is iRNA that targets MAST1 mRNA. In certain embodiments, the MAST1 inhibitor is shRNA designed to knock down gene expression. RNA interference initially discovered in plants as Post-Transcriptional Gene Silencing (PTGS), is a highly conserved mechanism triggered by double-stranded RNA (dsRNA) and able to down regulate transcript of genes homologous to the dsRNA. The dsRNA is first processed by Dicer into short duplexes of 21-23 nt, called short interfering RNAs (siRNAs). Incorporated in RNA-induced silencing complex (RISC), they are able to mediate gene silencing through cleavage of the target mRNA.

In some embodiments, any RNA inhibitor can be employed for any of the methods provided herein. Thus, any of siRNA, miRNA, dsRNA, shRNA, etc. can be employed in any of the methods provided herein as the MAST1 inhibitor.

“siRNA” or “small-interfering ribonucleic acid” refers to two strands of ribonucleotides which hybridize along a complementary region under physiological conditions. The siRNA molecules comprise a double-stranded region that is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. Methods to analyze and identify siRNAs with sufficient sequence identity to inhibit expression of a specific target sequence are known in the art. A suitable mRNA target region would be the coding region. Also suitable are untranslated regions, such as the 5′-UTR, the 3′-UTR, and splice junctions as long as the regions are unique to the mRNA target and not directed to an mRNA poly A tail.

The length of the region of the siRNA complementary to the target, in accordance with the present disclosure, may be from 15 to 100 nucleotides, 18 to 25 nucleotides, 20 to 23 nucleotides, or more than 15, 16, 17 or 18 nucleotides. Where there are mismatches to the corresponding target region, the length of the complementary region is generally required to be somewhat longer. In certain embodiments, the RNA capable of RNA interference comprises a human MAST1 sequence of 18 to 25 nucleotides or greater than 15, 16, 17, or 18 nucleotides. Homo sapiens microtubule associated serine/threonine kinase 1 (MAST1), mRNA NCBI Reference Sequence is NM_014975.2, hereby incorporated by reference. Alternatively, spliced transcript variants encoding distinct isoforms have been found for this gene.

Xu et al. report enhancing tumor cell response to chemotherapy through nanoparticle mediated co-delivery of siRNA and cisplatin prodrug. Proc Natl Acad Sci USA 2013, 110(46):18638-43.

He et al. report self-assembled nanoscale coordination polymers carrying siRNAs and cisplatin for effective treatment of resistant ovarian cancer. Biomaterials. 2015, 36:124-3.

Cho et al. report targeted delivery of siRNA-generating DNA nanocassettes using multifunctional nanoparticles. Small. 2013, 9(11):1964-73.

Since siRNA may be expressed from a RNA polymerase III (e.g., U6 or H1) promoter, a short hairpin siRNA (shRNA) gene may be cloned into expression vectors containing a polymerase III promoter to produce shRNAs from plasmid or viral vectors following transfecting into cells. See Brummelkamp et al., Science, 2002, 296, 550-553; Miyagishi & Taira, Nat. Biotechnol, 2002-497-500; McAnuff et al, J. Pharm. Sci. 2007, 96, 2922-2930; Bot et al., Blood, 2005, 106, 1147-1153. The shRNAs are further processed into siRNAs by a cellular endoribonuclease.

In certain embodiments, the disclosure relates to particles comprising a nucleic acid such as siRNA, DNA encoding for a siRNA, or siRNA expressing nanocassettes targeting MAST1. In certain embodiments, the disclosure relates to the particle further comprising a targeting ligand, e.g., shRNA, and a ligand that targets a receptor specifically expressed on tumor cells. In certain embodiments, the nanoparticles can be lipid particles, liposomes, lipoplexes, lipoids, polymers particles, cyclodextrin particles, Chitosan particles, polyethylene particles, gold particles, quantum dots (QDs) or iron oxide nanoparticles (IONPs). The particle may carry a chemotherapy drug. See Lee et al. Biomed Res Int. 2013, 2013:782041 entitled recent developments in nanoparticle based siRNA delivery for cancer therapy.

While not intending to be limited by theory, it is noted that it appears that lestaurtinib inhibits MAST1 as an ATP competitive inhibitor. Thus, in some embodiments, where designated, the function of lestaurtinib is to inhibit MAST1 as an ATP competitive inhibitor.

In some embodiments, the platinum-based chemotherapy agent is administered prior to administration of the MAST1 inhibitor. In some embodiments, the platinum-based chemotherapy agent is administered prior to administration of the MAST1 inhibitor. In some embodiments, the MAST1 inhibitor is administered prior to administration of the platinum-based chemotherapy agent. In some embodiments, the MAST1 inhibitor and the platinum-based chemotherapy agent are administered concurrently. In some embodiments, the MAST1 inhibitor and the platinum-based chemotherapy agent are administered at the same time.

In some embodiments, a kit is provided. The kit comprises an effective amount of a combination of a platinum-based chemotherapy agent and a microtubule associated serine/threonine-protein kinase (MAST) inhibitor. The effective amount is sufficient to provide a synergistic response in a subject who has a cancer that is resistant to the platinum-based chemotherapy agent without the MAST1 inhibitor. In some embodiments, the response is a sensitization of cells to cisplatin treatment so that their viability drops by at least 20% more than when treated without a MAST1 inhibitor.

### Examples

### MAST1 is Important for Cisplatin-Resistant Cancer Cell Proliferation and Tumor Growth

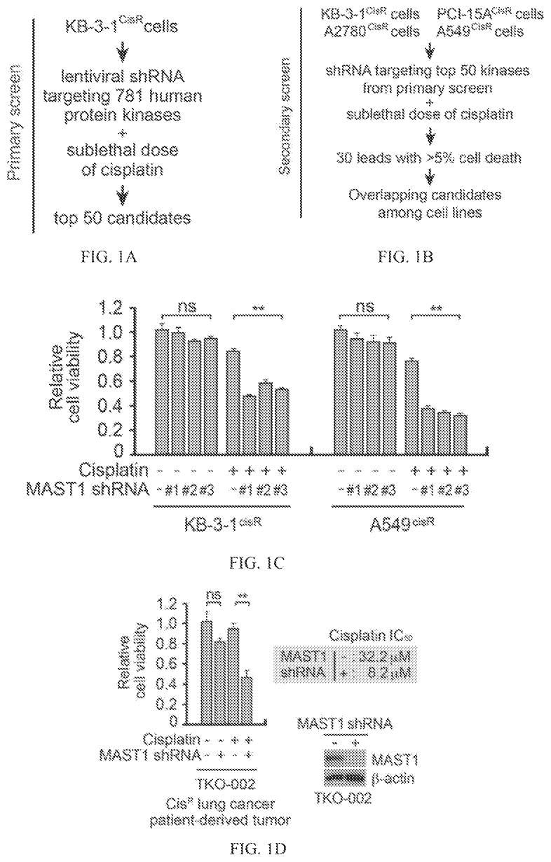

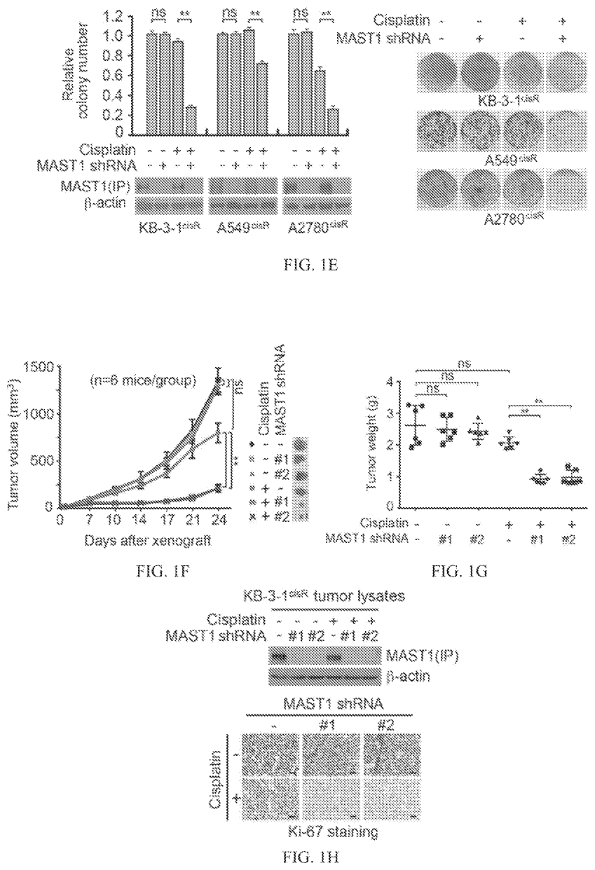

To gain insight into the role of protein kinase signaling in cancer chemoresistance, kinomewide RNAi screen was performed using a lentiviral shRNA library targeting 781 human kinase genes and kinase-related genes represented by 4,518 shRNA constructs (OpenBiosystems). The primary screen involved transducing cisplatin-resistant or taxol-resistant human carcinoma cell lines, KB-3-1^{cisR} and KB-3-1^{taxolR}, with a lentivirus pool containing shRNAs targeting each of the 781 individual genes, and treating with sublethal doses of cisplatin or taxol. From the primary screen using KB-3-1^{cisR}, 567 of 781 genes were selected for ranking by excluding shRNAs with low infection efficiency and shRNA clones that alone induced more than 15% cell death (FIG. 1A). The secondary screen evaluated the top 50 ranking candidate genes in four cisplatin-resistant cancer cell lines including head and neck squamous cell carcinoma (HNSCC) PCI-15A, lung cancer A549, and ovarian cancer A2780 in addition to KB-3-1 cells (FIG. 1B). Microtubule associated serine/threonine kinase 1 (MAST1) was identified as the third most effective target from the primary screen, and emerged as a lead hit from the secondary screen, as it sensitized cancer cells to cisplatin treatment across cancer types. MAST1 ranked 756 as an effective synthetic lethal target for taxol treatment, suggesting that targeting MAST1 specifically sensitizes cells to the chemotherapy agent cisplatin.

To confirm the screening results for MAST1, MAST1 stable knockdown cells were generated using individual shRNA clones. Targeting MAST1 with 3 different shRNA clones attenuated cell viability only in the presence of cisplatin in KB-3-1^{cisR} and A549^{cisR} cells (FIG. 1C). Moreover, knockdown of MAST1 sensitized freshly isolated tumor cells TKO-002 to cisplatin treatment (FIG. 1D). TKO-002 is derived from a patient with platinum-refractory small cell lung carcinoma (SCLC). Sensitization was not observed in cancer cells treated with taxol, demonstrating that the synthetic lethal effect is specific to cisplatin. In addition, MAST1 knockdown attenuated colony formation potential of cancer cell lines only in the presence of cisplatin (FIG. 1E). This was validated in vivo. Xenograft tumors derived from KB-3-1^{cisR} cells with MAST1 knockdown using two different shRNA clones were treated with cisplatin. The experiments showed dramatic decrease in tumor growth rate, tumor mass and tumor proliferation compared to tumors derived from cells with MAST1 knockdown without cisplatin treatment, or to tumors without MAST1 knockdown treated with cisplatin (FIGS. 1F-1H). These data suggest that MAST1 confers cisplatin resistance and targeting MAST1 sensitizes cancer cells to cisplatin by acting as a synthetic lethal partner.

MAST1 Confers Cisplatin Resistance Through cRaf Independent MEK1 Activation

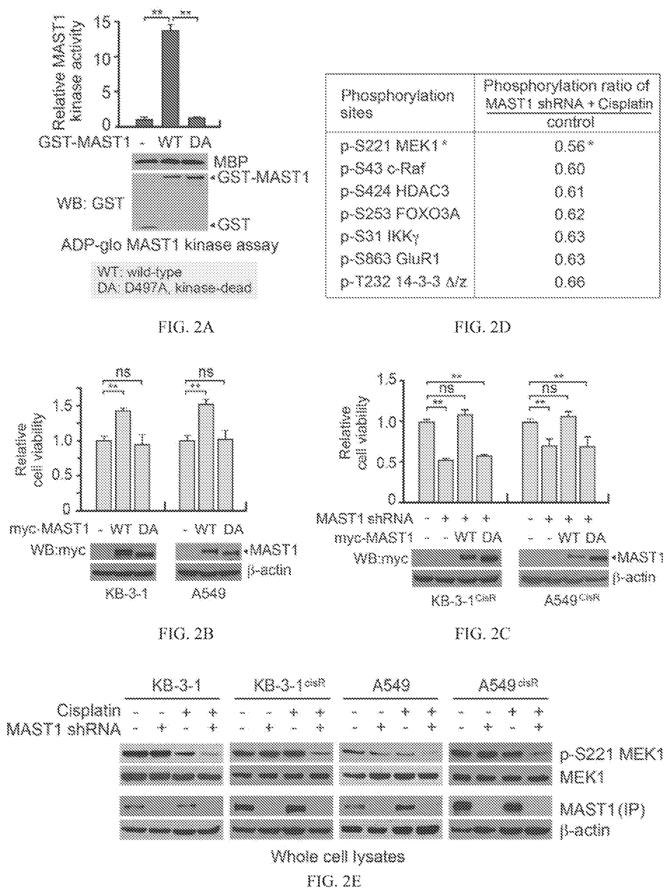

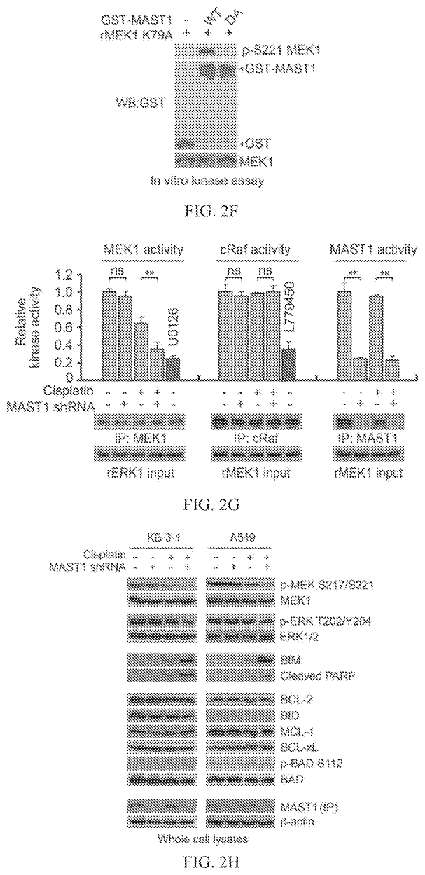

Whether the kinase activity of MAST1 is required to provide cisplatin resistance in cancer cells was tested. Thus, a kinase-dead mutant form of MAST1 was generated by mutating proton acceptor active residue aspartic acid (D) at 497 to alanine (A) in the corresponding wild type (WT) clone (UniProt). D497A mutation in MAST1 abolished its kinase activity (FIG. 2A). Overexpression of MAST1 WT but not DA (kinase-dead mutant D497A) conferred cisplatin resistance to cisplatin sensitive parental cells (FIG. 2B). Consistently, shRNA resistant-MAST1 WT but not DA rescued the cisplatin resistance attenuated due to MAST1 knockdown in cisplatin resistant cells (FIG. 2C). These data suggest that the kinase activity of MAST1 is required for cancer cells to acquire cisplatin-resistant pro-survival signals.

To gain insight into the role of MAST1 as a kinase in cisplatin resistance, high-throughput phosphorylation profiling was performed with 1,318 site-specific antibodies from over signaling pathways (Full Moon BioSystems). A spectrum of proteins was identified whose phosphorylation levels were decreased in KB-3-1^{cisR} cells only when MAST1 was knocked down and cells were treated with cisplatin (FIG. 2D). Among these, a decrease in inhibitory phosphorylation at S43 cRaf was observed. The greatest decrease was seen in phosphorylation of MEK1 at S221, which is involved in its activation Immunoblotting confirmed that knockdown of MAST1 together with cisplatin treatment in cancer cells did not affect the phosphorylation levels of STAT3 or AKT, but decreased the phosphorylation level of MEK1 (FIG. 2E). Moreover, MAST1 directly phosphorylates MEK1 at S221 in an in vitro MAST1 kinase assay (FIG. 2F). Supporting this finding, the kinase activity of MEK1 was significantly decreased, but cRaf activity was not altered, by cisplatin treatment and MAST1 knockdown in KB-3-1 cells (FIG. 2G). Targeting MEK1 by stable knockdown or its specific inhibitor U0126 sensitized cancer cells to cisplatin, although MEK1 knockdown itself attenuated cell growth, which was not observed with MAST1 knockdown. These results confirmed that MEK1 is a downstream effector of MAST1, which contributes to cisplatin resistance in cancer cells. Moreover, assessment of a group of apoptotic factors revealed that MEK1 and subsequent ERK inactivation upon MAST1 knockdown and cisplatin treatment specifically resulted in the accumulation of its downstream pro-apoptotic factor BIM (FIG. 2H). Furthermore, MAST1 knockdown together with sub-lethal doses of cisplatin resulted in enhanced apoptotic cell death in cancer cells (FIG. 2I). These data together suggest that MAST1 contributes to cisplatin resistance through a MEK1-mediated anti-apoptotic signaling pathway.

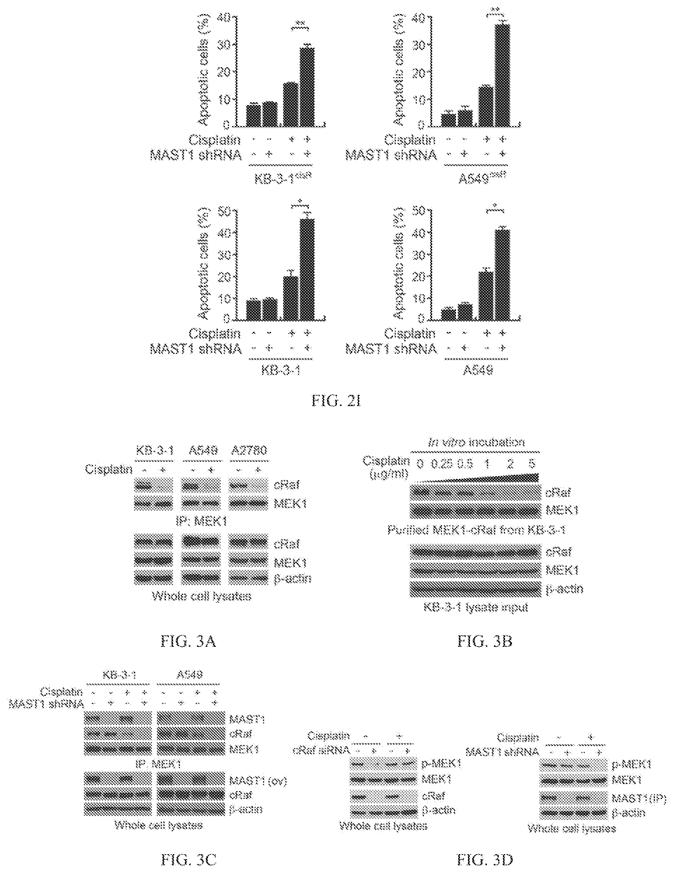

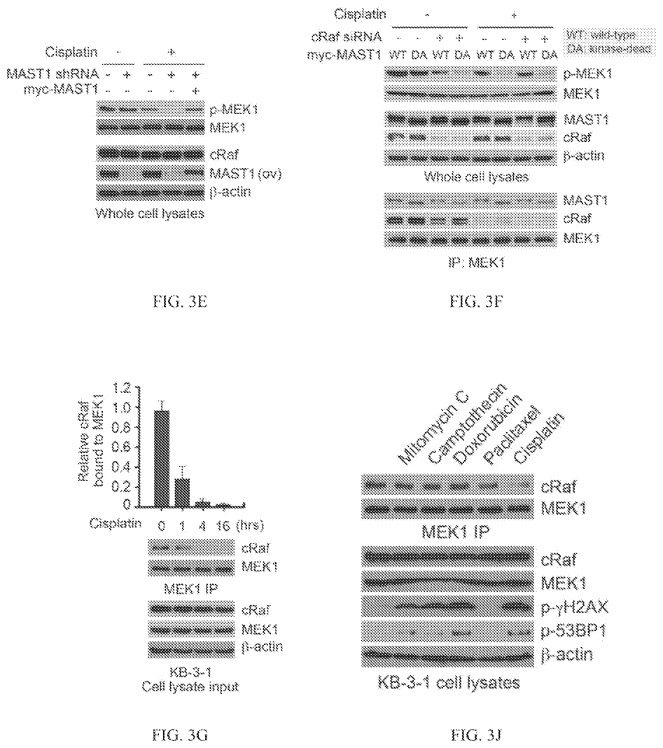

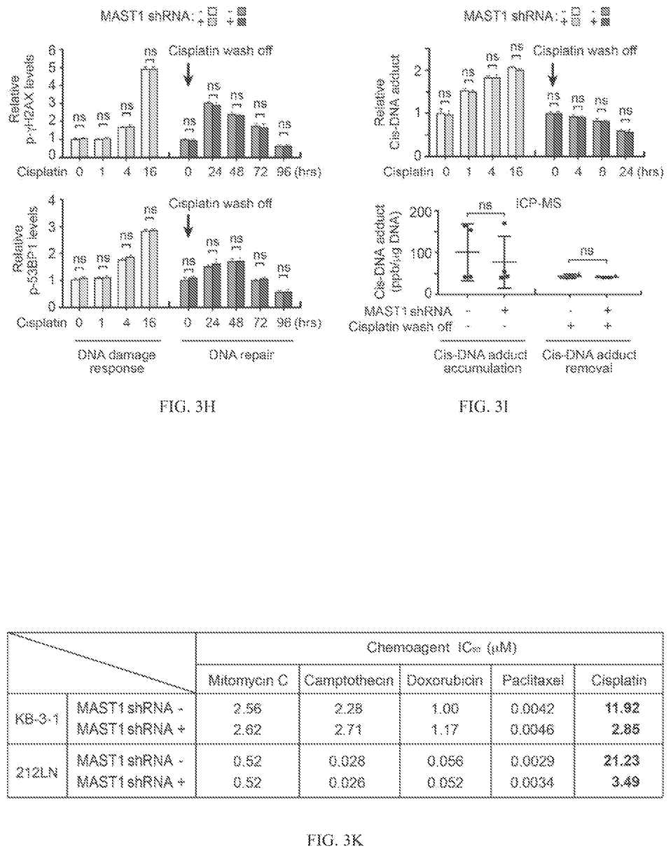

The molecular mechanism by which MAST1 controls MEK1 activation in the presence of cisplatin was evaluated. Interestingly, cisplatin treatment dissociates cRaf, the known upstream kinase of MEK, from MEK1 in diverse types of cancer cells (FIG. 3A). Moreover, cisplatin dissociates cRaf from MEK1 in a dose-dependent manner in vitro using purified MEK1-cRaf complex isolated from KB-3-1 cells by MEK1 immunoprecipitation, suggesting that cisplatin directly induces dissociation of cRaf from MEK1 (FIG. 3B). In addition, co-immunoprecipitation revealed that MAST1 forms a complex with cRaf-MEK1 and cisplatin dissociates cRaf from MEK1, but MAST1 remains within the complex (FIG. 3C). Knockdown of MAST1 further enhanced cRaf and MEK1 dissociation in the presence of cisplatin, while MEK1 knockdown did not influence the MAST1 and cRaf interaction, suggesting that multiple binding sites exist in the complex, and MAST1 is coupled with cRaf not through MEK1. Next, whether cisplatin impacts the dependency of MEK1 on MAST1 or cRaf was investigated. Knockdown of cRaf decreased MEK1 phosphorylation in the absence but not the presence of cisplatin (FIG. 3D). In contrast, MEK1 phosphorylation was decreased by MAST1 knockdown in the presence but not the absence of cisplatin (FIG. 3D), and the decreased phosphorylation of MEK1 was restored by rescue expression of MAST1 in MAST1 knockdown cells with cisplatin (FIG. 3E). Furthermore, in the presence of cisplatin, MAST1 WT but not MAST1 kinase-dead mutant DA induced MEK1 activation regardless of cRaf knockdown (FIG. 3F). These results suggest that cisplatin switches cRaf-dependent MEK1 activation to MAST1-dependence in cancer cells.

To further explore whether the cisplatin-mediated switch to MAST1 dependence is a result of a DNA damage response signaling, the timing of the switch and the impact of MAST1 on cisplatin-DNA adduct and on DNA damage and repair in time course analyses was examined cRaf dissociated from MEK1 upon cisplatin treatment within one hour, whereas cisplatin-induced DNA damage and repair was a later event independent of MAST1 (FIGS. 3G-3H). In addition, loss of MAST1 had no impact on cisplatin adduct accumulation and removal (FIG. 3I). Moreover, MAST1 knockdown did not influence the Fanconi anemia DNA repair pathway and DNA damage response signaling including ATR-Chk1 and ATM-Chk2 pathway. Only cisplatin, but not other DNA damaging agents or the chemotherapy agent paclitaxel, induced dissociation of cRaf-MEK1 (FIG. 3J). In line with this dissociation, knockdown of MAST1 only enhanced sensitivity to cisplatin but not to other DNA damaging agents or paclitaxel (FIG. 3K). These data together suggest that MAST1 contributes to cisplatin resistant cell survival not by acting as a critical element of the DNA damage response but by activating MEK-mediated anti-apoptotic signaling.

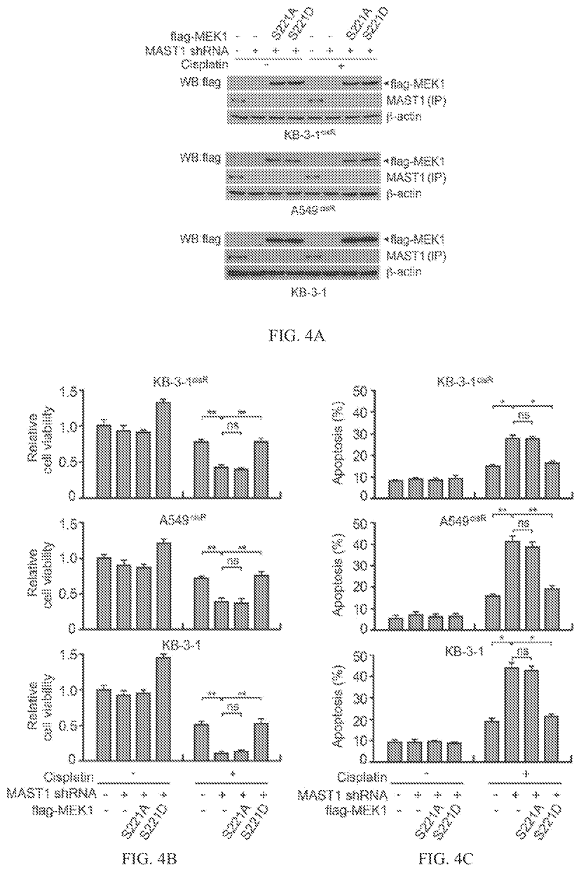

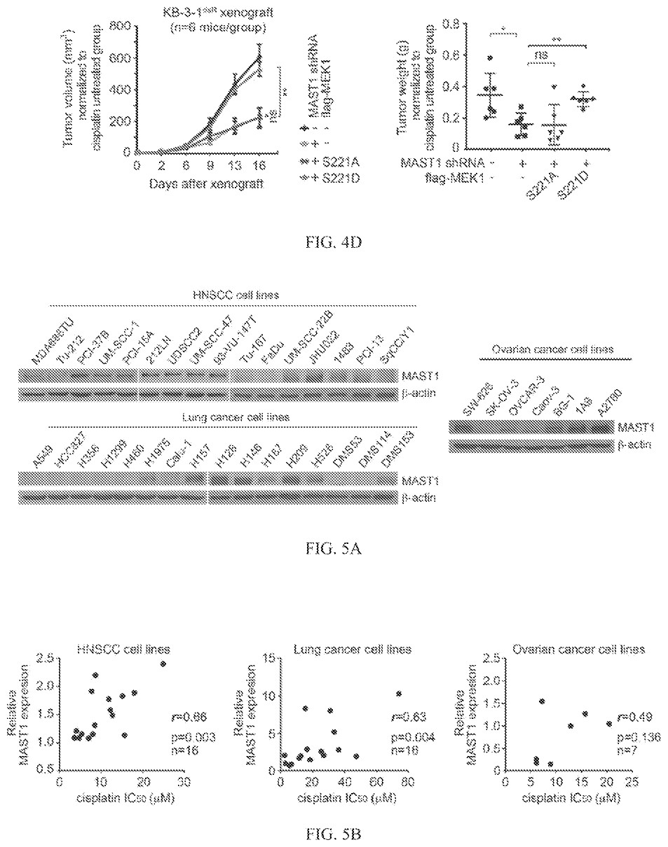

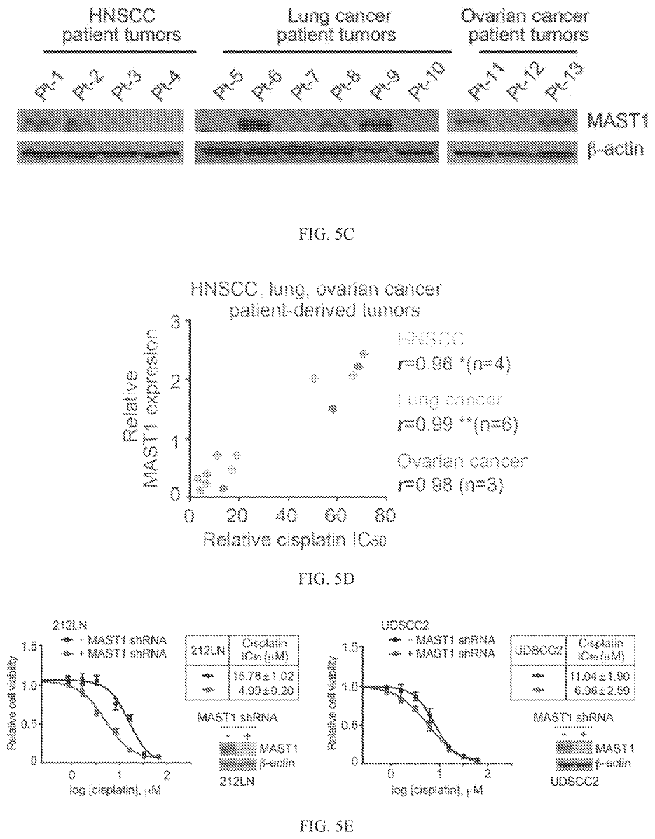

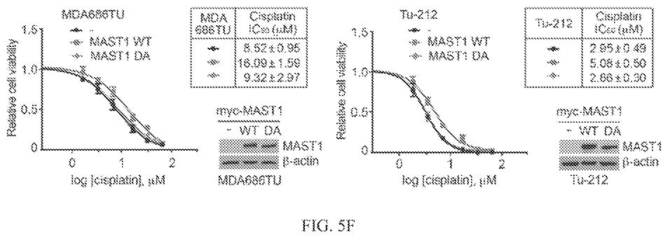

To demonstrate whether MEK1, as a downstream phosphorylation target of MAST1, contributes to MAST1-dependent pro-survival signals in response to cisplatin, cancer cell lines with stable knockdown of MAST1 and forced expression of phospho-mimetic or -deficient mutants of MEK1 were generated and examined for proliferative potential in vitro and in vivo (FIG. 4A). Silencing MAST1 using shRNA did not affect cell viability and apoptosis induction in the absence of cisplatin. With cisplatin treatment, knockdown of MAST1 significantly decreased cell viability and enhanced apoptosis induction, whereas expression of the MEK1 phospho-mimetic mutant S221D, but not the phospho-deficient mutant S221A, significantly rescued the phenotypes resulting from MAST1 knockdown (FIGS. 4B and 4C). Furthermore, S221D but not S221A MEK1 restored the attenuated tumor growth of MAST1 knockdown KB-3-1^{cisR} xenografts in the cisplatin-treated group (FIG. 4D). These results suggest that MAST1 signals through MEK1 by phosphorylation at serine 221 to promote cisplatin-resistant cancer cell proliferation and tumor growth.

MAST1 Expression Correlates with Cisplatin Resistance of Diverse Cancer Cell Lines and Primary Tumor Tissues

To investigate the expression of MAST1 and its relationship with cisplatin resistance in human cancer, MAST1 expression and cisplatin response were examined in 39 human cancer cell lines and 13 patient-derived tumors of HNSCC, lung cancer, and ovarian cancers. MAST1 expression positively correlates with cisplatin IC₅₀ (FIGS. 5A-5D). Knockdown of MAST1 by shRNA sensitized MAST1 expressing 212LN and UDSCC2 cells to cisplatin (FIG. 5E), In addition, overexpression of MAST1 WT but not kinase-dead mutant (DA) conferred cisplatin resistance in MDA686TU and Tu-212 cells that lack high expression of MAST1 (FIG. 5F). Furthermore, MAST1 expression levels, both protein and mRNA, were upregulated in cisplatin 25 resistant (cisR) cells that were chronically exposed to cisplatin, compared to parental cells.

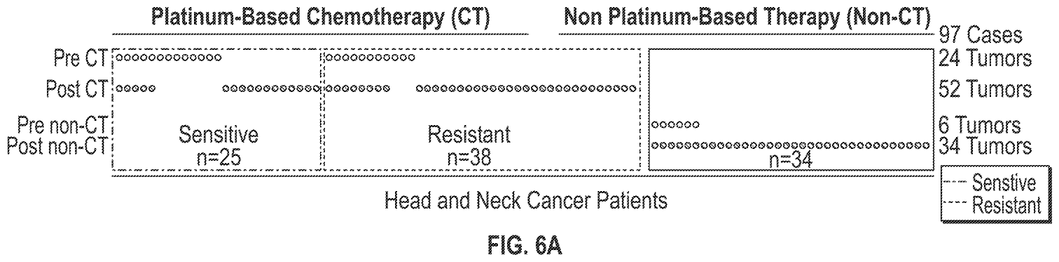

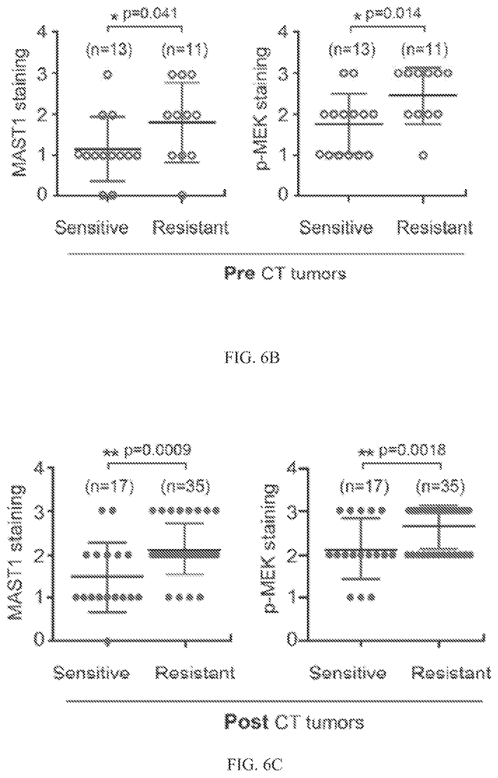

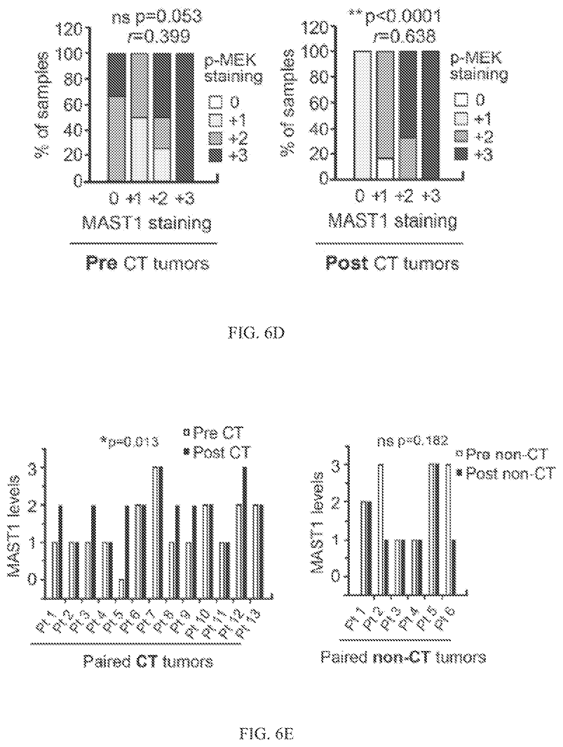

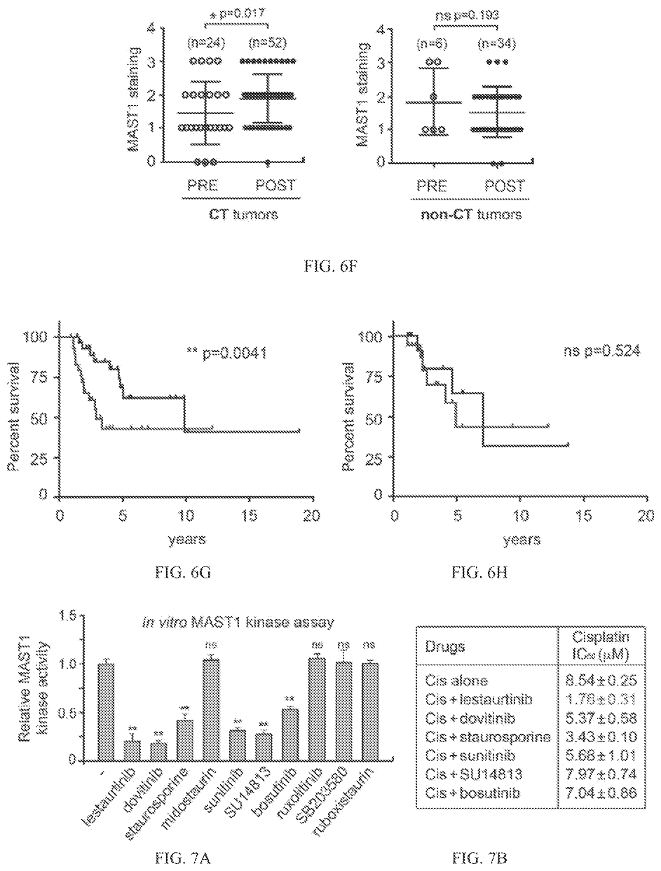

To demonstrate the clinical relevance of these findings, MAST1 and phospho-MEK levels were evaluated in 97 HNSCC patient cases who received platinum-containing therapy or nonplatinum based therapy such as radiation or surgery (FIG. 6A). For cases of platinum-containing therapy, patients who showed no evidence of disease for over 2 years after chemotherapy with cisplatin and/or carboplatin were considered ‘Sensitive’ and patients with disease recurrence within 2 years were considered ‘Resistant’. To determine whether an initially high MAST1 expression level confers cisplatin resistance, MAST1 and phospho-MEK levels were analyzed in tumor samples collected before platinum-containing chemotherapy. The ‘Resistant’ group showed significantly higher MAST1 and phospho-MEK1 levels compared to the ‘Sensitive’ group in pretreatment tumor samples (FIG. 6B). However, a more significant difference between ‘Sensitive’ and ‘Resistant’ groups were observed in post-treatment samples (FIG. 6C). In support of the finding that cisplatin drives MAST1-mediated MEK activation, significant positive correlation between MAST1 and phospho-MEK levels were observed in post-platinum treatment samples, but not in samples collected before the treatment (FIG. 6D). To determine whether MAST1 is induced during platinum treatment, pre- and post-treatment samples were evaluated. Analysis of pre- and post-treatment paired tumor samples obtained from individual patients showed that MAST1 staining increased after cisplatin treatment, whereas this increase was not observed in paired samples collected from cancer patients who received regimens other than platinum-based therapy (FIG. 6E). Similar to paired tumors, the analysis of non-paired pre- and post-treatment tumors also demonstrated that MAST1 was higher after platinum-based therapy, but not after non 15 platinum-based therapy (FIG. 6F). Furthermore, high MAST1 expression was associated with a worse clinical outcome only in cancer patients who received platinum-containing chemotherapy, but not in cancer patients who received non-platinum-based therapy (FIGS. 6G and 6H). Collectively, these results illustrate that initial MAST1 expression significantly correlates with platinum resistance (short term), and that MAST1 is further induced during platinum-based chemotherapy (long term), which together leads to platinum resistance and worse clinical outcome.

### Identification and Characterization of Lestaurtinib as a MAST1 Inhibitor to Target Cisplatin-Resistant Cancer

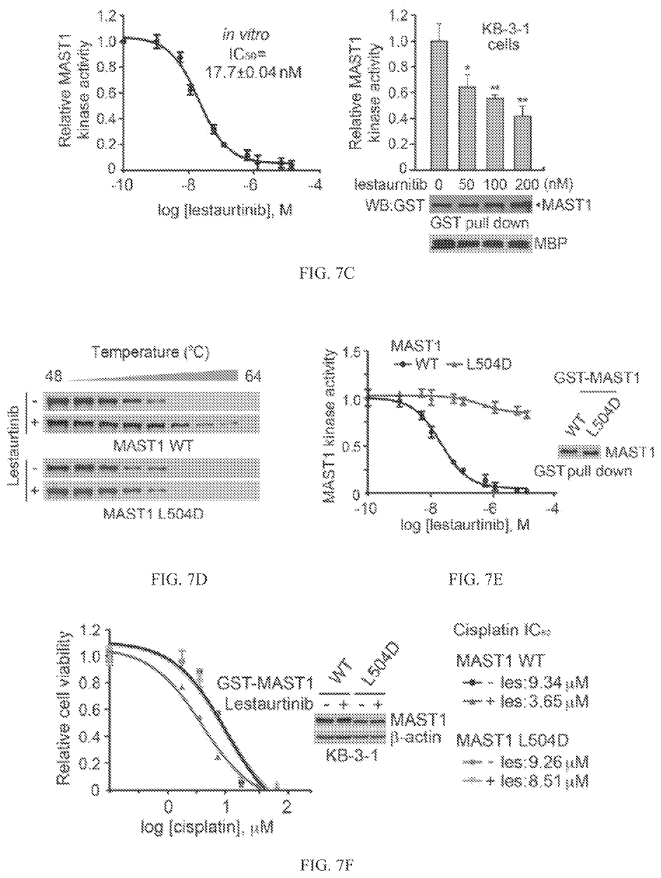

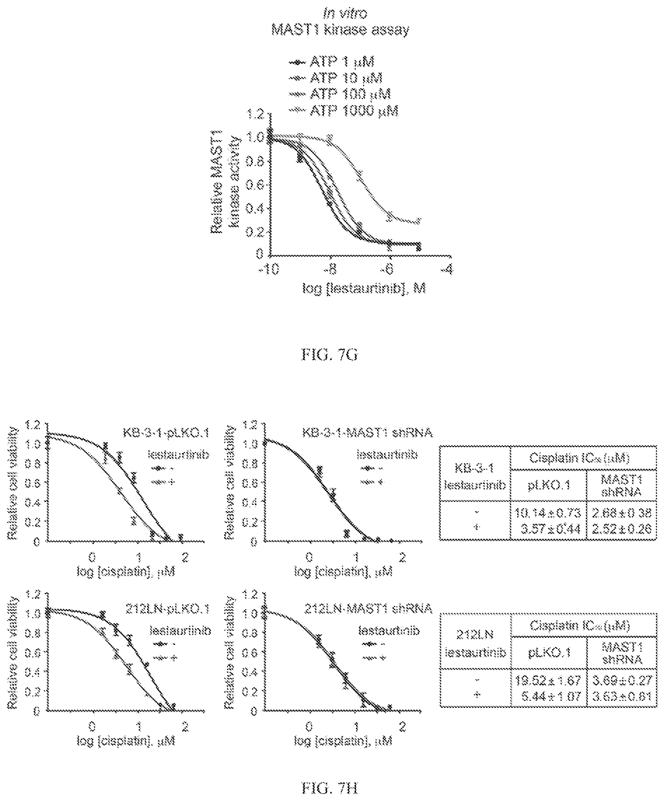

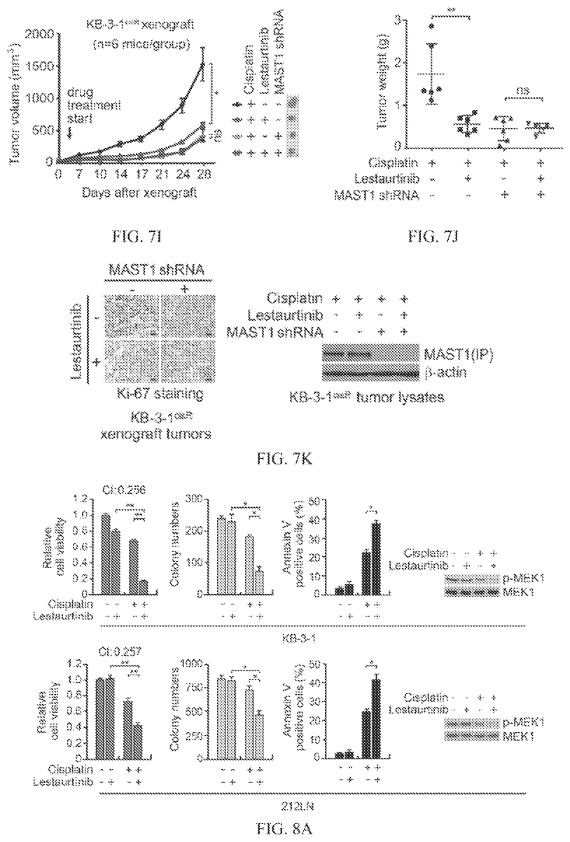

MAST1 is upregulated in cisplatin-resistant cancer and that attenuation of MAST1 sensitizes cancer cells to cisplatin treatment implicates MAST1 as a promising anti-cancer target to overcome cisplatin resistance. Potential MAST1 inhibitors were screened by testing the top small molecules among mature kinase inhibitors documented to bind to MAST1 (FIG. 7A), according to the IUPHAR database, which provides binding reactivity of the 72 inhibitors against 456 kinases (Davis et al., 2011). Six (6) inhibitors out of that showed a significant decrease in MAST1 activity were evaluated for their ability to sensitize cancer cells to cisplatin-induced cell death. Among these, lestaurtinib sensitized cells to cisplatin treatment the most using a concentration that attenuates cell viability by less than 20% when treated alone (FIG. 7B). Lestaurtinib effectively inhibited Ser/Thr kinase MAST1 in vitro and in cells (FIG. 7C). To demonstrate the selectivity of lestaurtinib binding to MAST1, a MAST1 L504D mutant was generated. Leucine 504 in MAST1 is predicted to be critical for lestaurtinib binding based on the structure of lestaurtinib and PRK1, a kinase that has similar catalytic domain to MAST1. Cellular thermal shift assay showed that MAST1 L504D mutant no longer binds to lestaurtinib (FIG. 7D). The kinase activity of the binding-deficient mutant MAST1 L504D and cisplatin resistance of cells carrying this mutant were unaltered by lestaurtinib (FIGS. 7E and 7F), suggesting that lestaurtinib sensitizes cells to cisplatin by binding and inhibiting MAST1 kinase activity. Lestaurtinib inhibits MAST1 as an ATP competitive inhibitor (FIG. 7G). Lestaurtinib is a mature tyrosine kinase inhibitor known to inhibit FLT3, JAK2, and Trk. Lestaurtinib sensitizes cells to cisplatin treatment, while the effect was abolished in MAST1 knockdown cells in vitro and in vivo in xenograft mice (FIGS. 7H-7K). Other known FLT3, Trk, or JAK2 inhibitors did not alter cisplatin sensitivity in cancer cells. Furthermore, FLT3 and Trk were not expressed in the cancer cells tested, and genetic inhibition of JAK2 did not alter cisplatin response. These data together suggest that cisplatin sensitivity conferred by lestaurtinib occurs mainly through MAST1 inhibition, not through other kinases. Our results demonstrate that lestaurtinib is a MAST1 inhibitor that sensitizes cancer cells to cisplatin.

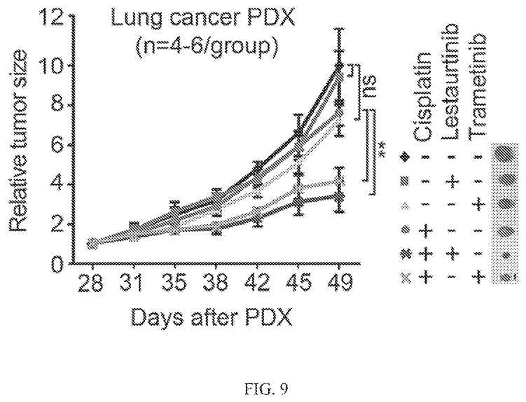

Finally, the efficacy of lestaurtinib in abrogating cisplatin resistance was evaluated in vitro and in vivo. Lestaurtinib in combination with cisplatin synergistically attenuated cell viability in KB-3-1 and 212LN cells with combination index (CI) of 0.256 and 0.257, respectively (FIG. 8A,). Similar results were obtained in colony formation assays (FIG. 8A). Consistent with the phenotype observed in MAST1 knockdown cells, inhibition of MAST1 by lestaurtinib in combination with cisplatin resulted in increased apoptotic cell death and decreased MEK activation (FIG. 8A). In addition, combination of lestaurtinib and cisplatin synergistically attenuated cell viability in primary platinum-refractory SCLC patient-derived tumor TKO-002 with CI value of 0.57 (FIG. 8B). Consistent with MAST1 knockdown, targeting MAST1 with lestaurtinib only sensitized cells to cisplatin but not to other DNA damaging agents or chemotherapy agents, further supporting that the synergistic effect of MAST1 inhibition is specific to cisplatin. Moreover, the effect of lestaurtinib was greater in lines with higher MAST1 levels in a panel of cancer cell lines and patient-derived tumors. Lastly, patient-derived xenograft (PDX) models were established using diverse cancer patient tumors including HNSCC, lung cancer, and ovarian cancer. The dosage of 20 mg/kg lestaurtinib and 5 mg/kg cisplatin did not induce significant changes in body weight, histopathology, or hematopoietic properties. The combination significantly reduced PDX tumor growth and tumor cell proliferation compared to single agent treatment (FIGS. 8C and 8D). Lestaurtinib significantly attenuated MAST1 activity and MEK1 phosphorylation in PDX tumors (FIGS. 8E and 8F). Finally, to demonstrate whether the effect of MAST1 inhibitor lestaurtinib on the response to cisplatin therapy is mediated through MEK, the effect of a MEK inhibitor on enhancing cisplatin therapy was compared to that of lestaurtinib in an additional PDX model of lung cancer. The combination of trametinib and cisplatin mimicked the lestaurtinib and cisplatin effect resulting in attenuated PDX tumor growth and proliferation, but lestaurtinib showed slightly greater synergistic effect than trametinib (FIG. 9). These results together suggest that lestaurtinib is a MAST1 inhibitor with promising anti-tumor effect in combination therapy with cisplatin in human cancers.

| SEQ ID NO. 1: | |
|---|---|
| 1 | msdslwtals nfsmpsfpgg smfrrtkscr tsnrkslilt stsptlprph splpghlgss |
| 61 | pldsprnfsp ntpahfsfas srradgrrws laslpssgyg tntpsstvss scssqerlhq |
| 121 | lpyqptvdel hflskhfgst esitdedggr rspayrprsr slspgrspss ydneivmmnh |
| 181 | vykerfpkat aqmeeklrdf trayepdsvl pladgvlsfi hhqiielard cltksrdgli |
| 241 | ttvyfyelqe nlekllqday erseslevaf vtqlvkklli iisrparlle clefnpeefy |
| 301 | hlleaaegha keghlvktdi pryiirqlgl trdpfpdvvh leeqdsggsn tpeqddlseg |
| 361 | rsskakkppg endfdtikli sngaygavyl vrhrdtrqrf amkkinkqnl ilrnqiqqaf |
| 421 | verdiltfae npfvvgmfcs fetrrhlcmv meyveggdca tllknigalp vemarmyfae |
| 481 | tvlaleylhn ygivhrdlkp dnllitsmgh ikltdfglsk mglmslttnl yeghiekdar |
| 541 | efldkqvcgt peyiapevil rqgygkpvdw wamgiilyef lvgcvpffgd tpeelfgqvi |
| 601 | sddilwpegd ealpteaqll issllqtnpl vrlgaggafe vkqhsffrdl dwtgllrqka |
| 661 | efiphlesed dtsyfdtrsd ryhhvnsyde ddtteeepve irqfsscspr fskvyssmeq |
| 721 | lsqhepktpv aaagsskrep stkgpeekva gkreglgglt lrektwrggs peikrfsase |
| 781 | asflegeasp plgarrrfsa llepsrfsap qededearlr rpprpssdpa gsldarapke |
| 841 | etqgegtssa gdseatdrpr pgdlcppskd gdasgpratn dlvlrrarhq qmsgdvavek |
| 901 | rpsrtggkvi ksasatalsv mipavdphgs splaspmspr slssnpssrd sspsrdyspa |
| 961 | vsglrspiti qrsgkkygft lrairvymgd tdvysvhhiv whveeggpaq eaglcagdli |
| 1021 | thvngepvhg mvhpevveli lksgnkvavt ttpfentsir igparrssyk akmarrnkrp |
| 1081 | sakegqeskk rsslfrkitk qsnllhtsrs lsslnrslss sdslpgspth glparspths |
| 1141 | yrstpdsayl gassqssspa sstpnspass ashhirpstl hglspklhrq yrsarcksag |
| 1201 | niplsplaht psptqasppp lpghtvgssh ttqsfpaklh ssppvvrprp ksaepprspl |
| 1261 | lkrvqsaekl gaslsadkkg alrkhslevg hpdfrkdfhg elalhslaes dgetppvegl |
| 1321 | gaprqvavrr lgrqesplsl gadpllpega srppvsskek espggaeact pprattpggr |
| 1381 | tlerdvgctr hqsvqtedgt ggmaravaka alspvqehet grrsssgeag tplvpivvep |
| 1441 | arpgakavvp qplgadskgl qepaplapsv peaprgrerw vlevveertt lsgprskpas |
| 1501 | pklspepqtp slapakcsap ssavtpvppa sllgsgtkpq vgltsrcpae avppagltkk |
| 1561 | gvsspappgp |

1. A method of treating cancer in a subject, the method comprising:
administering to the subject an effective amount of a platinum-based chemotherapy agent in combination with a microtubule associated serine/threonine-protein kinase 1 (MAST1) inhibitor.

2. The method of claim 1, wherein the platinum-based chemotherapy agent is selected from cisplatin, carboplatin, oxaliplatin, phenanthriplatin, nedaplatin, triplatin tetranitrate, picoplatin, pyriplatin, lipoplatin, or satraplatin.

3. The method of claim 1, wherein the MAST1 inhibitor is an antibody, small molecule compound, peptide, or siRNA.

4. The method of claim 3, wherein the small molecule compound is lestaurtinib or a derivative, prodrug, or salt thereof.

5. The method of claim 1, wherein the cancer is selected from testicular cancer, ovarian cancer, cervical cancer, breast cancer, bladder cancer, head and neck cancer, esophageal cancer, lung cancer, mesothelioma, brain tumors, or neuroblastoma.

6. The method of claim 1, wherein the method further comprises administering an additional anti-cancer agent.

7. The method of claim 6, wherein the additional anti-cancer agent is alemtuzumab, trastuzumab, ibritumomab tiuxetan, brentuximab vedotin, temozolomide, ado-trastuzumab emtansine, denileukin diftitox, blinatumomab, interferon alpha, aldesleukin, carmustine, bevacizumab, procarbazine, lomustine, vincristine, gefitinib, erlotinib, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin, rituximab, and/or lenalidomide or combinations thereof.

8. The method of claim 1, wherein the subject is administered a combination of microtubule associated serine/threonine-protein kinase 1 (MAST1) inhibitor, a taxane, and a platinum-based chemotherapy agent.

9. The method of claim 8, wherein the taxane is paclitaxel, taxol, docetaxel, or combinations thereof.

10. The method of claim 1, wherein the subject is administered a combination of two or more of lestaurtinib, paclitaxel, and cisplatin.

11. The method of claim 1, wherein the platinum-based chemotherapy agent is administered concurrently with the MAST1 inhibitor.

12. The method of claim 1, wherein the MAST1 inhibitor is administered prior to administering the platinum-based chemotherapy agent.

13. A method for treating cancer in a subject, the method comprising: a) obtaining a sample from the subject, wherein the subject has been administered a platinum-based chemotherapy agent; b) detecting MAST1 protein or encoding nucleic acid in the sample from the subject; and c) when the detected MAST1 protein or encoding nucleic acid is higher than a control, administering to the subject an effective amount of a microtubule associated serine/threonine protein kinase 1 (MAST1) inhibitor or other kinase inhibitor alone or in combination with the platinum-based chemotherapy agent.

14. The method of claim 13, wherein the control amount is determined from a cancerous sample considered sensitive to a platinum-based chemotherapy agent.

15. The method of claim 13, wherein the MAST1 inhibitor is an antibody, small molecule compound, peptide, or siRNA.

16. The method of claim 15, wherein the small molecule compound is lestaurtinib or a derivative, prodrug, or salt thereof.

17. The method of claim 13, further comprising administering an additional anti-cancer agent.

18. A pharmaceutical composition comprising an effective amount of a platinum-based chemotherapy agent in combination with a microtubule associated serine/threonine-protein kinase 1 (MAST1) inhibitor for a cancer that is resistant to the platinum-based chemotherapy agent without the MAST1 inhibitor.

19. A use of a microtubule associated serine/threonine-protein kinase 1 (MAST1) inhibitor in treatment of a subject with a platinum-based chemotherapeutic resistant cancer.

20. A kit comprising an effective amount of a combination of a platinum-based chemotherapy agent and a microtubule associated serine/threonine-protein kinase 1 (MAST1) inhibitor, wherein the effective amount is sufficient to provide a synergistic response in a subject who has a cancer that is resistant to the platinum-based chemotherapy agent without the MAST1 inhibitor.

21. A composition comprising a MAST1 inhibitor and a platinum-based chemotherapy agent.


SEQUENCE LISTING (1 sequence)

SEQ ID NO: 1 - protein, 1570 aa, Homo sapiens
MSDSLWTALSNFSMPSFPGGSMFRRTKSCRTSNRKSLILTSTSPTLPRPHSPLPGHLGSSPLDSPRNFSPNTPAHFSFASSRRADGRRWSLASLPSSGYGTNTPSSTVSSSCSSQERLHQLPYQPTVDELHFLSKHFGSTESITDEDGGRRSPAVRPRSRSLSPGRSPSSYDNEIVMMNHVYKERFPKATAQMEEKLRDFTRAYEPDSVLPLADGVLSFIHHQIIELARDCLTKSRDGLITTVYFYELQENLEKLLQDAYERSESLEVAFVTQLVKKLLIIISRPARLLECLEFNPEEFYHLLEAAEGHAKEGHLVKTDIPRYIIRQLGLTRDPFPDVVHLEEQDSGGSNTPEQDDLSEGRSSKAKKPPGENDFDTIKLISNGAYGAVYLVRHRDTRQRFAMKKINKQNLILRNQIQQAFVERDILTFAENPFVVGMFCSFETRRHLCMVMEYVEGGDCATLLKNIGALPVEMARMYFAETVLALEYLHNYGIVHRDLKPDNLLITSMGHIKLTDFGLSKMGLMSLTTNLYEGHIEKDAREFLDKQVCGTPEYIAPEVILRQGYGKPVDWWAMGIILYEFLVGCVPFFGDTPEELFGQVISDDILWPEGDEALPTEAQLLISSLLQTNPLVRLGAGGAFEVKQHSFFRDLDWTGLLRQKAEFIPHLESEDDTSYFDTRSDRYHHVNSYDEDDTTEEEPVEIRQFSSCSPRFSKVYSSMEQLSQHEPKTPVAAAGSSKREPSTKGPEEKVAGKREGLGGLTLREKTWRGGSPEIKRFSASEASFLEGEASPPLGARRRFSALLEPSRFSAPQEDEDEARLRRPPRPSSDPAGSLDARAPKEETQGEGTSSAGDSEATDRPRPGDLCPPSKDGDASGPRATNDLVLRRARHQQMSGDVAVEKRPSRTGGKVIKSASATALSVMIPAVDPHGSSPLASPMSPRSLSSNPSSRDSSPSRDYSPAVSGLRSPITIQRSGKKYGFTLRAIRVYMGDTDVYSVHHIVWHVEEGGPAQEAGLCAGDLITHVNGEPVHGMVHPEVVELILKSGNKVAVTTTPFENTSIRIGPARRSSYKAKMARRNKRPSAKEGQESKKRSSLFRKITKQSNLLHTSRSLSSLNRSLSSSDSLPGSPTHGLPARSPTHSYRSTPDSAYLGASSQSSSPASSTPNSPASSASHHIRPSTLHGLSPKLHRQYRSARCKSAGNIPLSPLAHTPSPTQASPPPLPGHTVGSSHTTQSFPAKLHSSPPVVRPRPKSAEPPRSPLLKRVQSAEKLGASLSADKKGALRKHSLEVGHPDFRKDFHGELALHSLAESDGETPPVEGLGAPRQVAVRRLGRQESPLSLGADPLLPEGASRPPVSSKEKESPGGAEACTPPRATTPGGRTLERDVGCTRHQSVQTEDGTGGMARAVAKAALSPVQEHETGRRSSSGEAGTPLVPIVVEPARPGAKAVVPQPLGADSKGLQEPAPLAPSVPEAPRGRERWVLEVVEERTTLSGPRSKPASPKLSPEPQTPSLAPAKCSAPSSAVTPVPPASLLGSGTKPQVGLTSRCPAEAVPPAGLTKKGVSSPAPPGP